(19) United States
(12) Patent Application Publication
(10) Pub. No.: US 2026/0056105 A1
(43) Pub. Date: Feb. 26, 2026

(54) SYSTEMS AND METHODS FOR LIVE DETERMINATION OF FLUID ENERGY CONTENT

(71) Applicant: MICRO MOTION, INC., Boulder, CO (US)

(72) Inventor: George Alexander MACDONALD, Winnersh (GB)

(73) Assignee: MICRO MOTION, INC., Boulder, CO (US)

(21) Appl. No.: 19/374,577

(22) Filed: Oct. 30, 2025

Related U.S. Application Data
(63) Continuation of application No. 17/640,730.

(51) Int. Cl. G01N 9/36; G01N 9/00

(52) U.S. Cl. CPC G01N 9/36; G01N 9/002; G01N 2009/006

(57) ABSTRACT

A method for determining an inferential relationship between an inferred energy content and at least one measured quantity is disclosed. The inferential relationship yields an inferred energy content. The method uses a computer (200) having a processor (210) configured to execute commands based on data stored in a memory (220), the processor (210) implementing steps of an inference module (204) stored in the memory (220), the method comprising a step of determining, by the inference module (204) the inferential relationship by analyzing a relationship between known measurements of at least one measured energy content of at least one fluid and at least one corresponding measured value of a same type as the at least one measured quantity wherein the inferential relationship has a density term (B), wherein one of the at least one measured quantity is a measured density (ρ) and the density term (B) has an inverse density (1/ρ), the density term (B) representing an inverse relationship between density (p) and the inferred energy content, and wherein the measured density (ρ) is not a density of air (ρₐᵢᵣ).

### CROSS-REFERENCE TO RELATED APPLICATIONS

This application is a continuation of U.S. application Ser. No. 17/640,730, which is the National Stage of International Application No. PCT/US2019/050145, filed September 9,2019.

### TECHNICAL FIELD

The embodiments described below relate to determining properties of flow fluids, more particularly, to determining properties of flow fluids with varying compositions.

### BACKGROUND

Determining energy content of a flow fluid dynamically in a system where the composition of the fluid is expected to change is a challenging problem. Existing systems for measuring energy content of flow fluids are often cumbersome and difficult to deploy in settings where live measurements are required.

The energy content of a flow fluid often affects the financial value of the flow fluid for instance, in gas and oil applications. Common metrics of energy content include, for instance, Calorific Value (hereinafter, “CV”) and Wobbe index. Energy content metrics, including the Wobbe index, can be readily determined from CV using methods existing in the art, so the specification emphasizes the use of CV as a metric for energy content. CV can be expressed in units of kilojoules per kilogram (i.e. “by mass”) or units of kilojoules per standard cubic meter (at base conditions of 20° C. and 1.013 bar). Other systems of units are contemplated, for instance, British thermal units per pound may be used instead of kilojoules per kilogram, and British thermal units per cubic foot may be used instead of kilojoules per standard cubic meter. This specification is not limited to determination of CV, and any other energy content metric can be determined or derived from the CV. CV may be determined in a number of ways. One known equation for CV is the AGA5 equation, presented as Eq. (1):

CV=[(1571.5×SG)+144]−(25.318×% CO₂+16.639×% N₂) (1)
Here, SG is specific gravity, % CO₂ is percent carbon dioxide composition by volume, and % N₂ is percent nitrogen composition by volume. The equation presented accounts for the most major inert contributors, carbon dioxide and nitrogen gases, but further substances in a flow fluid may be considered, for instance, oxygen, helium, carbon monoxide, hydrogen sulfide, water (perhaps vapor), and hydrogen. Coefficients for the AGA 5 equation for these less prominent substances have been determined and are well-established in the art, but they have been omitted for purposes of brevity. Eq. (1) yields CV values in units of British thermal units per cubic foot.

One known system for direct determination of energy content is burning a fuel in a calorimeter and measuring the energy released. Few existing systems can apply these measurements live, and, if used in live gas lines can be dangerous. Also, live measurements with in-line systems still suffer from delays in the process of combusting and measuring. Some methods would have the fuel removed from a line and used in a calorimeter that does not have a live feed from a system. These methods suffer from delays in determinations of energy content for having to wait for sampling, combustion, and time to take measurements.

Another method for determining energy content is determining the composition of the fuel and then determining an overall energy content value based on a composition weighted average of the calorific values of each component of the composition. This is difficult to accomplish live or in-line because it is difficult to determine the composition of a flowing fluid as it flows. Also, in a system where the composition of the flowing fluid changes, there will be delays associated with determining the fluid composition, frustrating live energy content determinations.

Another set of methods used for determining energy content is a set of inferential methods. These methods have the benefit of being able to use live measurements to infer a value of interest. The inferential methods that exist suffer from inaccuracy and/or problems with determining some factors considered. For instance, many require knowledge of the thermal conductivity or heat capacity. In applications where composition of a flow fluid varies with time, as is common in gas and oil applications, compositions need to be determined in order to derive live measurements.

Existing systems also suffer from a reliance on a direct relationship between a measured density and a corresponding determined CV. When modeling the CV as having a direct relationship with density, it can be appreciated that the relationship has elements that appear to show an inverse relationship between a variable and the CV. Also, many methods suffer from having a term in the CV determination in which measured viscosity values are multiplied with measured density values. Further, while existing methods typically use temperature dependencies to determine measured pressure and viscosity, the methods do not account for temperature and/or pressure dependency of coefficients for measured density and measured viscosity terms. Still further, these temperature and/or pressure dependent terms do not have constant values that can be attributed to determinations for certain classifications of gases.

Accordingly, there is a need for systems and methods for quick inferential determination of energy content from quantities that can be measured live.

### SUMMARY

A method for determining an inferential relationship between an inferred energy content and at least one measured quantity is disclosed. The inferential relationship yields an inferred energy content. The method uses a computer (200) having a processor (210) configured to execute commands based on data stored in a memory (220), the processor (210) implementing steps of an inference module (204) stored in the memory (220), the method comprising a step of determining, by the inference module (204), the inferential relationship by analyzing a relationship between known measurements of at least one measured energy content of at least one fluid and at least one corresponding measured value of a same type as the at least one measured quantity wherein the inferential relationship has a density term (B), wherein one of the at least one measured quantity is a measured density (ρ) and the density term (B) has an inverse density (1/ρ), the density term (B) representing an inverse relationship between density (ρ) and the inferred energy content, and wherein the measured density (ρ) is not a density of air (ρₐᵢᵣ).

A method for using an inferential relationship between an inferred energy content and at least one measured quantity of a fluid is disclosed. The predetermined inferential relationship yields an inferred energy content. The method uses a computer (200) having a processor (210) configured to execute commands based on data stored in a memory (220), the processor (210) implementing steps of an inference module (204) stored in the memory (220). The method comprises steps of receiving, by the inference module (204), at least one measured value of a type of the at least one measured quantity and inferring, by the inference module (204), the inferred energy content from the inferential relationship and the at least one measured quantity, wherein the inferential relationship has a density term (B) and one of the at least one measured value is a measured density (ρ) and the density term (B) has an inverse density (1/ρ), the density term (B) representing an inverse relationship between the measured density (ρ) and the inferred energy content and wherein the measured density (ρ) is not a density of air (ρₐᵢᵣ).

An apparatus for using an inferential relationship between an inferred energy content and at least one measured quantity of a fluid is disclosed. The inferential relationship yields an inferred energy content. The apparatus has a computer (200) having a processor (210) configured to execute commands based on data stored in a memory (220), the processor (210) implementing steps of an inference module (204) stored in the memory (220), the inference module (204) configured to receive at least one measured value of a type of the at least one measured quantity and infer the inferred energy content from the inferential relationship and the at least one measured quantity, wherein the inferential relationship has a density term (B) and one of the at least one measured value is a measured density (ρ) and the density term (B) has an inverse density (1/ρ), the density term (B) representing an inverse relationship between the measured density (ρ) and the inferred energy content and wherein the measured density (ρ) is not a density of air (ρₐᵢᵣ).

An apparatus for determining an inferential relationship between an inferred energy content and at least one measured quantity is disclosed. The inferential relationship yields an inferred energy content. The apparatus has a computer (200) having a processor (210) and a memory (220), the processor (210) configured to execute commands based on data stored in the memory (220), the processor (210) executing an inference module (204) stored in the memory (220), the inference module (204) configured to determine the inferential relationship by analyzing a relationship between known measurements of at least one measured energy content of at least one fluid and at least one corresponding measured value of a same type as the at least one measured quantity, wherein the inferential relationship has a density term (B), wherein one of the at least one measured quantity is a measured density (ρ) and the density term (B) has an inverse density (1/ρ), the density term (B) representing an inverse relationship between density (ρ) and the inferred energy content and wherein the measured density (ρ) is not a density of air (ρₐᵢᵣ).

### ASPECTS

According to an aspect, a method for determining an inferential relationship between an inferred energy content and at least one measured quantity is disclosed. The inferential relationship yields an inferred energy content. The method uses a computer (200) having a processor (210) configured to execute commands based on data stored in a memory (220), the processor (210) implementing steps of an inference module (204) stored in the memory (220), the method comprising a step of determining, by the inference module (204), the inferential relationship by analyzing a relationship between known measurements of at least one measured energy content of at least one fluid and at least one corresponding measured value of a same type as the at least one measured quantity wherein the inferential relationship has a density term (B), wherein one of the at least one measured quantity is a measured density (ρ) and the density term (B) has an inverse density (1/ρ), the density term (B) representing an inverse relationship between density (ρ) and the inferred energy content, and wherein the measured density (ρ) is not a density of air (ρₐᵢᵣ).

Preferably, the inference module (204) does not account for any of viscosity (η), specific gravity, and the density of air (ρₐᵢᵣ) in the density term (B).

Preferably, the inference module (204) determines the inferential relationship without accounting for any of a heat capacity, a thermal conductivity, a dielectric constant, a refractive index, a thermal diffusivity, a laminar resistance, and a turbulent resistance.

Preferably, another of the at least one measured quantity is a measured viscosity (η), the inferential relationship further comprising a shift term (A) and a viscosity term (C), the viscosity term (C) accounting for the measured viscosity (η).

Preferably, the inferential relationship is a sum of the shift term (A), the density term (B), and the viscosity term (C).

Preferably, the viscosity term (C) has a viscosity (η), the viscosity term (C) representing a direct relationship between viscosity (η) and the inferred energy content.

Preferably, the at least one measured value further comprises a measured temperature (T) and a measured pressure (P) wherein the shift term (A) comprises a corresponding temperature and pressure dependent shift term coefficient (k₁(P,T)), the density term (B) comprises a corresponding temperature and pressure dependent density term coefficient (k₂(P,T)), and the viscosity term (C) comprises a corresponding temperature and pressure dependent viscosity term coefficient (k₃(P,T)).

Preferably, the density term (B) is the density term coefficient (k₂(P,T)) multiplied by the inverse density (1/ρ).

Preferably, the viscosity term (C) is the viscosity term coefficient (k₃(P,T)) multiplied by the viscosity (η).

Preferably, the shift term (A) is the shift term coefficient (k₁(P,T)).

Preferably, wherein the inferential relationship is represented by the equation,

$$CV = {k}_{1} ( P , T ) + {k}_{2} ( P , T ) \times \frac{1}{ρ} + {k}_{3} ( P , T ) \times η .$$

Preferably, the shift term coefficient (k₁(P,T)), the density term coefficient (k₂(P,T)), and viscosity term coefficient (k₃(P,T)) are derived using corresponding coefficient constants (e.g. a₁-a₄, b₁-b₄, c₁-c₄, d₁-d₄) associated with the at least one fluid.

Preferably, the shift term coefficient (k₁(P,T)) is dependent upon a relationship between the measured pressure (P), the measured temperature (T), and at least one shift coefficient constant (e.g. a₁-a₄) of the coefficient constants (e.g. a₁-a₄, b₁-b₄, c₁-c₄, d₁-d₄), the density term coefficient (k₂(P,T)) is dependent upon a relationship between the measured pressure (P), the measured temperature (T) and at least one density coefficient constant (e.g. b₁-b₄) of the coefficient constants (e.g. a₁-a₄, b₁-b₄, c₁-c₄, d₁-d₄), and the viscosity term coefficient (k₃(P,T)) is dependent upon a relationship between the measured pressure (P), the measured temperature (T) and at least one viscosity coefficient constant (e.g. c₁-c₄) of the coefficient constants (e.g. a₁-a₄, b₁-b₄, c₁-c₄, d₁-d₄).

Preferably, the relationship between the measured pressure (P), the measured temperature (T), and the at least one shift coefficient constant (e.g. a₁-a₄) is represented by the equation, k₁(P,T)=[a₁+a₂(T−20)]+[a₃+a₄(T−20)]×P, the relationship between the measured pressure (P), the measured temperature (T) and the at least one density coefficient constant (e.g. b₁-b₄) is represented by the equation, k₂(P,T)=[b₁+b₂(T−20)]+[b₃+b₄(T−20)]×P, and the relationship between measured pressure (P), the measured temperature (T), and the at least one viscosity coefficient constant (e.g. c₁-c₄) is represented by the equation, k₃(P,T)=[c₁+c₂(T−20)]+[c₃+c₄(T−20)]×P.

Preferably, the inferential relationship further comprises an inert term (D), the inert term accounting for a percent composition of carbon dioxide (% CO₂), the inert term (D) having a temperature (T) and pressure (P) dependent inert term coefficient (k₄(P,T)), wherein the inert term coefficient (k₄(P,T)) is determined using inert term coefficient constants (e.g. d1-d4).

Preferably, inert term coefficient (k₄(P,T)) is determined from the relationship, k₄(P,T)=[d₁+d₂(T−20)]+[d₃+d₄(T−20)]×P, the inferential relationship being

$$CV = {k}_{1} ( P , T ) + {k}_{2} ( P , T ) \times \frac{1}{ρ} + {k}_{3} ( P , T ) \times η + {k}_{4} ( P , T ) \times \% {CO}_{2} .$$

Preferably, the analyzing, by the inference module (204), further comprises associating the coefficient constants (e.g. a₁-a₄, b₁-b₄, c₁-c₄, d₁-d₄) with at least one class of fluids of which one or more of the at least one fluid is a member.

Preferably, the at least one class of fluids is one or more of fuel gas, natural gas, flare gas, liquefied natural gas, biogas, shale gas, and a class of fluid associated with a geographic region.

Preferably, the inferential relationship may be characterized by the coefficient constants (e.g. a₁-a₄, b₁-b₄, c₁-c₄, d₁-d₄) such that the coefficient constants may be used as predetermined coefficient constants (e.g. a₁-a₄, b₁-b₄, c₁-c₄, d₁-d₄) in a live inferential determination of an inferred energy content that is determined live while taking live measurements of the same type as the at least one measured value.

Preferably, the inferred energy content is an inferred calorific value.

According to an aspect, a method for using an inferential relationship between an inferred energy content and at least one measured quantity of a fluid is disclosed. The predetermined inferential relationship yields an inferred energy content. The method uses a computer (200) having a processor (210) configured to execute commands based on data stored in a memory (220), the processor (210) implementing steps of an inference module (204) stored in the memory (220). The method comprises steps of receiving, by the inference module (204), at least one measured value of a type of the at least one measured quantity and inferring, by the inference module (204), the inferred energy content from the inferential relationship and the at least one measured quantity, wherein the inferential relationship has a density term (B) and one of the at least one measured value is a measured density (ρ) and the density term (B) has an inverse density (1/ρ), the density term (B) representing an inverse relationship between the measured density (ρ) and the inferred energy content and wherein the measured density (ρ) is not a density of air (ρₐᵢᵣ).

Preferably, the inference module (204) does not account for any of viscosity (η), specific gravity, and the density of air (ρₐᵢᵣ) in the density term (B).

Preferably, the inference module (204) infers the inferred energy content without accounting for any of a heat capacity, a thermal conductivity, a dielectric constant, a refractive index, a thermal diffusivity, a laminar resistance, and a turbulent resistance.

Preferably, another of the at least one measured value is a measured viscosity (η), the inferential relationship further comprising a shift term (A) and a viscosity term (C), the viscosity term (C) accounting for the measured viscosity (η).

Preferably, the inferential relationship is a sum of the shift term (A), the density term (B), and the viscosity term (C).

Preferably, the viscosity term (C) has a viscosity (η), the viscosity term (C) representing a direct relationship between viscosity (η) and the inferred energy content. Preferably, at least one measured value further comprises a measured temperature (T) and a measured pressure (P) wherein the shift term (A) comprises a corresponding temperature and pressure dependent shift term coefficient (k₁(P,T)), the density term (B) comprises a corresponding temperature and pressure dependent density term coefficient (k₂(P,T)), and the viscosity term (C) comprises a corresponding temperature and pressure dependent viscosity term coefficient (k₃(P,T)).

Preferably, the density term (B) is the density term coefficient (k₂(P,T)) multiplied by the inverse density (1/ρ).

Preferably, the viscosity term (C) is the viscosity term coefficient (k₃(P,T)) multiplied by the viscosity (η).

Preferably, the shift term (A) is the shift term coefficient (k₁(P,T)).

Preferably, the inferential relationship is represented by the equation,

$$CV = {k}_{1} ( P , T ) + {k}_{2} ( P , T ) \times \frac{1}{ρ} + {k}_{3} ( P , T ) \times η .$$

Preferably, the shift term coefficient (k₁(P,T)), the density term coefficient (k₂(P,T)), and viscosity term coefficient (k₃(P,T)) are evaluated using corresponding predetermined coefficient constants (e.g. a₁-a₄, b₁-b₄, c₁-c₄, d₁-d₄) associated with the fluid.

Preferably, the shift term coefficient (k₁(P,T)) is evaluated, by the inference module (204), using a relationship between the measured pressure (P), the measured temperature (T), and at least one predetermined shift coefficient constant (e.g. a₁-a₄) of the predetermined coefficient constants (e.g. a₁-a₄, b₁-b₄, c₁-c₄, d₁-d₄), the density term coefficient (k₂(P,T)) is evaluated, by the inference module (204), using a relationship between the measured pressure (P), the measured temperature (T) and at least one predetermined density coefficient constant (e.g. b₁-b₄) of the predetermined coefficient constants (e.g. a₁-a₄, b₁-b₄, c₁-c₄, d₁-d₄), and the viscosity term coefficient (k₃(P,T)) is evaluated, by the inference module (204), using a relationship between the measured pressure (P), the measured temperature (T) and at least one predetermined viscosity coefficient constant (e.g. c₁-c₄) of the predetermined coefficient constants (e.g. a₁-a₄, b₁-b₄, c₁-c₄, d₁-d₄).

Preferably, the relationship between the measured pressure (P), the measured temperature (T) and the at least one predetermined shift coefficient constant (e.g. a₁-a₄) is represented by the equation, k₁(P,T)=[a₁+a₂(T−20)]+[a₃+a₄(T−20)]×P, the relationship between the measured pressure (P), the measured temperature (T) and the at least one predetermined density coefficient constant (e.g. b₁-b₄) is represented by the equation, k₂(P, T)=[b₁+b₂(T—20)]+[b₃+b₄(T−20)]×P, the relationship between the measured pressure (P), the measured temperature (T) and the at least one predetermined viscosity coefficient constant (e.g. c₁-c₄) is represented by the equation, k₃(P,T)=[c₁+c₂(T−20)]+[c₃+c₄(T−20)]×P.

Preferably, the at least one measured value further comprises a measured inert content, wherein the measured inert content is a percent composition of carbon dioxide by volume (% CO₂), the inferential relationship further having an inert term (D), the inert term (D) accounting for the percent composition of carbon dioxide (% CO₂), the inert term (D) having a temperature (T) and pressure (P) dependent inert term coefficient (k₄(P,T)), wherein the inert term coefficient (k₄(P,T)) is determined using inert term coefficient constants (e.g. d1-d4).

Preferably, the inert term coefficient (k₄(P,T)) is determined from the relationship, k₄(P,T)=[d₁+d₂(T−20)]+[d₃+d₄(T−20)]×P, the inferential relationship being

$$CV = {k}_{1} ( P , T ) + {k}_{2} ( P , T ) \times \frac{1}{ρ} + {k}_{3} ( P , T ) \times η + {k}_{4} ( P , T ) \times \% {CO}_{2} .$$

Preferably, the inferring, by the inference module (204), comprises inferring the inferred energy content of the fluid using predetermined coefficient constants (e.g. a₁-a₄, b₁-b₄, c₁-c₄, d₁-d₄) associated with at least one class of fluids of which one or more of the at least one fluid is a member.

Preferably, the at least one class of fluids is one or more of fuel gas, natural gas, flare gas, liquefied natural gas, biogas, shale gas, and a class of fluid associated with a geographic region.

Preferably, the method further comprises steps of measuring, by a vibratory sensor (5), at least one raw data signal, while the fluid is interacting with the vibratory sensor (5), supplying, by the vibratory sensor (5), the at least one raw data signal to a measurement module (202), and processing, by the measurement module (202), the at least one raw data signal to determine data representing one or more of the at least one measured value, wherein the receiving, by the inference module (204) comprises receiving the data representing the one or more of the at least one measured value from the measurement module (202).

Preferably, the one or more of the at least one measured value comprises the measured density (ρ).

Preferably, the method further comprises measuring, by a pressure sensor (150), the measured pressure (P), wherein the receiving, by the inference module (204), comprises receiving the measured pressure (P).

Preferably, one or more of the measured temperature (T) and the measured pressure (P) is assumed to be consistent.

Preferably, the inferred energy content is a calorific value.

According to an aspect, an apparatus for using an inferential relationship between an inferred energy content and at least one measured quantity of a fluid is disclosed. The inferential relationship yields an inferred energy content. The apparatus has a computer (200) having a processor (210) configured to execute commands based on data stored in a memory (220), the processor (210) implementing steps of an inference module (204) stored in the memory (220), the inference module (204) configured to receive at least one measured value of a type of the at least one measured quantity and infer the inferred energy content from the inferential relationship and the at least one measured quantity, wherein the inferential relationship has a density term (B) and one of the at least one measured value is a measured density (ρ) and the density term (B) has an inverse density (1/ρ), the density term (B) representing an inverse relationship between the measured density (ρ) and the inferred energy content and wherein the measured density (ρ) is not a density of air (ρₐᵢᵣ).

Preferably, the inference module (204) does not account for any of viscosity (η), specific gravity, and the density of air (ρₐᵢᵣ) in the density term (B).

Preferably, the inference module (204) infers the inferred energy content without accounting for any of heat capacity, thermal conductivity, a dielectric constant, a refractive index, thermal diffusivity, a laminar resistance, and a turbulent resistance.

Preferably, another of the at least one measured value is a measured viscosity (η), the inferential relationship further comprising a shift term (A) and a viscosity term (C), the viscosity term (C) accounting for the measured viscosity (η).

Preferably, the inferential relationship is a sum of the shift term (A), the density term (B), and the viscosity term (C).

Preferably, the viscosity term (C) has a viscosity (η), the viscosity term (C) representing a direct relationship between viscosity (η) and the inferred energy content.

Preferably, the at least one measured value further comprises a measured temperature (T) and a measured pressure (P) wherein the shift term (A) comprises a corresponding temperature and pressure dependent shift term coefficient (k₁(P,T)), the density term (B) comprises a corresponding temperature and pressure dependent density term coefficient (k₂(P,T)), and the viscosity term (C) comprises a corresponding temperature and pressure dependent viscosity term coefficient (k₃(P,T)).

Preferably, the density term (B) is the density term coefficient (k₂(P,T)) multiplied by the inverse density (1/ρ).

Preferably, the viscosity term (C) is the viscosity term coefficient (k₃(P,T)) multiplied by the viscosity (η).

Preferably, the shift term (A) is the shift term coefficient (k₁(P,T)).

Preferably, the inferential relationship is represented by the equation,

$$CV = {k}_{1} ( P , T ) + {k}_{2} ( P , T ) \times \frac{1}{ρ} + {k}_{3} ( P , T ) \times η .$$

Preferably, the shift term coefficient (k₁(P,T)), the density term coefficient (k₂(P,T)), and viscosity term coefficient (k₃(P,T)) are evaluated using corresponding predetermined coefficient constants (e.g. a₁-a₄, b₁-b₄, c₁-c₄, d₁-d₄) associated with the fluid.

Preferably, wherein the shift term coefficient (k₁(P,T)) is evaluated, by the inference module (204), using a relationship between the measured pressure (P), the measured temperature (T), and at least one predetermined shift coefficient constant (e.g. a₁-a₄) of the predetermined coefficient constants (e.g. a₁-a₄, b₁-b₄, c₁-c₄, d₁-d₄), the density term coefficient (k₂(P,T)) is evaluated, by the inference module (204), using a relationship between the measured pressure (P), the measured temperature (T) and at least one predetermined density coefficient constant (e.g. b₁-b₄) of the predetermined coefficient constants (e.g. a₁-a₄, b₁-b₄, c₁-c₄, d₁-d₄), and the viscosity term coefficient (k₃(P,T)) is evaluated, by the inference module (204), using a relationship between the measured pressure (P), the measured temperature (T) and at least one predetermined viscosity coefficient constant (e.g. c₁-c₄) of the predetermined coefficient constants (e.g. a₁-a₄, b₁-b₄, c₁-c₄, d₁-d₄).

Preferably, the relationship between the measured pressure (P), the measured temperature (T) and the at least one predetermined shift coefficient constant (e.g. a₁-a₄) is represented by the equation, k₁(P,T)=[a₁+a₂(T−20)]+[a₃+a₄(T−20)]×P, the relationship between the measured pressure (P), the measured temperature (T) and the at least one predetermined density coefficient constant (e.g. b₁-b₄) is represented by the equation, k₂(P, T)=[b₁+b₂(T−20)]+[b₃+b₄(T−20)]×P, the relationship between the measured pressure (P), the measured temperature (T) and the at least one predetermined viscosity coefficient constant (e.g. c₁-c₄) is represented by the equation, k₃(P,T)=[c₁+c₂(T−20)]+[c₃+c₄(T−20)]×P.

Preferably, the inferential relationship further comprises an inert term (D), the inert term accounting for a percent composition of carbon dioxide (% CO₂), the inert term (D) having a temperature (T) and pressure (P) dependent inert term coefficient (k₄(P,T)), wherein the inert term coefficient (k₄(P,T)) is determined using inert term coefficient constants (e.g. d1-d4).

Preferably, the inert term coefficient (k₄(P,T)) is determined from the relationship, k₄(P,T)=[d₁+d₂(T−20)]+[d₃+d₄(T−20)]×P, the inferential relationship being

$$CV = {k}_{1} ( P , T ) + {k}_{2} ( P , T ) \times \frac{1}{ρ} + {k}_{3} ( P , T ) \times η + {k}_{4} ( P , T ) \times \% {CO}_{2} .$$

Preferably, the inferring, by the inference module (204), comprises inferring the inferred energy content of the fluid using predetermined coefficient constants (e.g. a₁-a₄, b₁-b₄, c₁-c₄, d₁-d₄) associated with at least one class of fluids of which one or more of the at least one fluid is a member.

Preferably, the at least one class of fluids is one or more of fuel gas, natural gas, flare gas, liquefied natural gas, shale gas, biogas, and a class of fluids from a geographic region.

Preferably, one or more of the measured temperature (T) and the measured pressure (P) is a constant, based on a determination, by the inference module (204), that the one or more of the measured temperature (T) and the measured pressure (P) is sufficiently consistent in operating conditions.

Preferably, the apparatus further comprises a measurement module (202) stored in the memory (220), the measurement module configured to receive, by the measurement module (202), at least one raw data signal and process, by the measurement module (202), the at least one raw data signal to determine data representing one or more of the at least one measured value, wherein the receiving, by the inference module (204), comprises receiving the data representing the one or more of the at least one measured value from the measurement module (202).

Preferably, the apparatus is a vibratory sensor (5), the apparatus configured to interact with the fluid, wherein the computer (200) is a meter electronics (20) configured to determine one or more of the at least one measured value based on measurements taken by the vibratory sensor (5).

Preferably, the apparatus comprises a first tine (104a) and a second tine (104b) that interact with the fluid, a driver (102) that receives a drive signal from the computer (200), and drives a motion in the first tine (104a) based on the drive signal, a response sensor (106) configured to generate a response signal representing a responsive motion of the second tine (104b) and transmit the response signal to the meter electronics (20), wherein the meter electronics (20) is configured to determine the one or more of the at least one measured value from one or more of the drive signal and the response signal.

Preferably, the one or more of the at least one measured value comprises the measured density (ρ).

Preferably, the inferred energy content is a calorific value.

According to an aspect, an apparatus for determining an inferential relationship between an inferred energy content and at least one measured quantity is disclosed. The inferential relationship yields an inferred energy content. The apparatus has a computer (200) having a processor (210) and a memory (220), the processor (210) configured to execute commands based on data stored in the memory (220), the processor (210) executing an inference module (204) stored in the memory (220), the inference module (204) configured to determine the inferential relationship by analyzing a relationship between known measurements of at least one measured energy content of at least one fluid and at least one corresponding measured value of a same type as the at least one measured quantity, wherein the inferential relationship has a density term (B), wherein one of the at least one measured quantity is a measured density (ρ) and the density term (B) has an inverse density (1/ρ), the density term (B) representing an inverse relationship between density (ρ) and the inferred energy content and wherein the measured density (ρ) is not a density of air (ρₐᵢᵣ).

Preferably, the inference module (204) does not account for any of viscosity (η), specific gravity, and the density of air (ρₐᵢᵣ) in the density term (B).

Preferably, the inference module (204) determines the inferential relationship without accounting for any of heat capacity, thermal conductivity, a dielectric constant, a refractive index, thermal diffusivity, a laminar resistance, and a turbulent resistance.

Preferably, another of the at least one measured quantity is a measured viscosity (η), the inferential relationship further comprising a shift term (A) and a viscosity term (C), the viscosity term (C) accounting for the measured viscosity (η).

Preferably, the inferential relationship is a sum of the shift term (A), the density term (B), and the viscosity term (C).

Preferably, the viscosity term (C) has a viscosity (η), the viscosity term (C) representing a direct relationship between viscosity (η) and the inferred energy content.

Preferably, the at least one measured value further comprises a measured temperature (T) and a measured pressure (P) wherein the shift term (A) comprises a corresponding temperature and pressure dependent shift term coefficient (k₁(P,T)), the density term (B) comprises a corresponding temperature and pressure dependent density term coefficient (k₂(P,T)), and the viscosity term (C) comprises a corresponding temperature and pressure dependent viscosity term coefficient (k₃(P,T)).

Preferably, the density term (B) is the density term coefficient (k₂(P,T)) multiplied by the inverse density (1/ρ).

Preferably, the viscosity term (C) is the viscosity term coefficient (k₃(P,T)) multiplied by the viscosity (η).

Preferably, the shift term (A) is the shift term coefficient (k₁(P,T)).

Preferably, the inferential relationship is represented by the equation,

$$CV = {k}_{1} ( P , T ) + {k}_{2} ( P , T ) \times \frac{1}{ρ} + {k}_{3} ( P , T ) \times η .$$

Preferably, the shift term coefficient (k₁(P,T)), the density term coefficient (k₂(P,T)), and viscosity term coefficient (k₃(P,T)) are derived using corresponding coefficient constants (e.g. a₁-a₄, b₁-b₄, c₁-c₄, d₁-d₄) associated with the at least one fluid.

Preferably, the shift term coefficient (k₁(P,T)) is dependent upon a relationship between the measured pressure (P), the measured temperature (T), and at least one shift coefficient constant (e.g. a₁-a₄) of the coefficient constants (e.g. a₁-a₄, b₁-b₄, c₁-c₄, d₁-d₄), the density term coefficient (k₂(P,T)) is dependent upon a relationship between the measured pressure (P), the measured temperature (T) and at least one density coefficient constant (e.g. b₁-b₄) of the coefficient constants (e.g. a₁-a₄, b₁-b₄, c₁-c₄, d₁-d₄), and the viscosity term coefficient (k₃(P,T)) is dependent upon a relationship between the measured pressure (P), the measured temperature (T) and at least one viscosity coefficient constant (e.g. c₁-c₄) of the coefficient constants (e.g. a₁-a₄, b₁-b₄, c₁-c₄, d₁-d₄).

Preferably, the relationship between the measured pressure (P), the measured temperature (T), and the at least one shift coefficient constant (e.g. a₁-a₄) is represented by the equation, k₁(P,T)=[a₁+a₂(T−20)]+[a₃+a₄(T−20)]×P, the relationship between the measured pressure (P), the measured temperature (T) and the at least one density coefficient constant (e.g. b₁-b₄) is represented by the equation, k₂(P,T)=[b₁+b₂(T−20)]+[b₃+b₄(T−20)]×P, and the relationship between measured pressure (P), the measured temperature (T), and the at least one viscosity coefficient constant (e.g. c₁-c₄) is represented by the equation, k₃(P,T)=[c₁+c₂(T−20)]+[c₃+c₄(T−20)]×P.

Preferably, the inferential relationship further comprises an inert term (D), the inert term accounting for a percent composition of carbon dioxide (% CO₂), the inert term (D) having a temperature (T) and pressure (P) dependent inert term coefficient (k₄(P,T)), wherein the inert term coefficient (k₄(P,T)) is determined using inert term coefficient constants (e.g. d1-d4).

Preferably, the inert term coefficient (k₄(P,T)) is determined from the relationship, k₄(P,T)=[d₁+d₂(T−20)]+[d₃+d₄(T−20)]×P, the inferential relationship being

$$CV = {k}_{1} ( P , T ) + {k}_{2} ( P , T ) \times \frac{1}{ρ} + {k}_{3} ( P , T ) \times η + {k}_{4} ( P , T ) \times \% {CO}_{2} .$$

Preferably, the analyzing, by the inference module (204), further comprises associating the coefficient constants (e.g. a₁-a₄, b₁-b₄, c₁-c₄, d₁-d₄) with at least one class of fluids of which one or more of the at least one fluid is a member.

Preferably, the at least one class of fluids is one or more of fuel gas, natural gas, flare gas, liquefied natural gas, shale gas, biogas, and a class of gases associated with a geographic region.

Preferably, the inferential relationship may be characterized by the coefficient constants (e.g. a₁-a₄, b₁-b₄, c₁-c₄, d₁-d₄) such that the coefficient constants may be used as predetermined coefficient constants (e.g. a₁-a₄, b₁-b₄, c₁-c₄, d₁-d₄) in a live inferential determination of an inferred energy content that is determined live while taking live measurements of the same type as the at least one measured value.

Preferably, the apparatus is a vibratory sensor (5) and the computer (200) is a meter electronics (20).

Preferably, the apparatus determines one or more of the at least one measured value and the apparatus provides the one or more of the at least one measured value to the inference module (204) for use in the inferring of the inferred energy content.

Preferably, the one or more of the at least one measured value comprises the measured density (ρ) and the measured viscosity (η).

Preferably, the inferred energy content is an inferred calorific value.

### BRIEF DESCRIPTION OF THE DRAWINGS

The same reference number represents the same element on all drawings. It should be understood that the drawings are not necessarily to scale.

FIG. 1 shows a block diagram of an embodiment of a system 100 for determining the energy content of a flow fluid.

FIG. 2 shows a block diagram of an embodiment of a computer 200.

FIG. 3 shows a flowchart of an embodiment of a method 300 for using an inferential relationship between measured parameters and fluid energy content.

FIG. 4 shows a flowchart of an embodiment of a method 400 for determining an inferential relationship between measured parameters and flow fluid energy content.

FIG. 5 shows a flowchart of another embodiment of a method 500 for determining an inferential relationship between measured parameters and flow fluid energy content.

FIG. 6 shows a flowchart of still another embodiment of a method 600 for determining an inferential relationship between measured parameters and flow fluid energy content.

FIG. 7 shows a flowchart of an embodiment of a method 700 for inferring an energy content from measured parameters.

FIG. 8 shows a flowchart of another embodiment of a method 800 for inferring an energy content from measured parameters.

FIG. 9 shows a flowchart of still another embodiment of a method 900 for inferring an energy content from measured parameters.

FIG. 10 shows a graph 1000 of an embodiment of a comparison between inferred energy content values derived using mass units and energy content determined from direct methods.

FIG. 11 shows a graph 1100 of an embodiment of error in the inferred calorific values relative to the directly determined calorific values.

FIG. 12 shows a graph 1200 of an embodiment of a comparison between inferred energy content values inferred at standard conditions and energy content determined from direct methods.

FIG. 13 shows a graph 1300 of an embodiment of error in the inferred calorific values relative to the directly determined calorific values.

### DETAILED DESCRIPTION

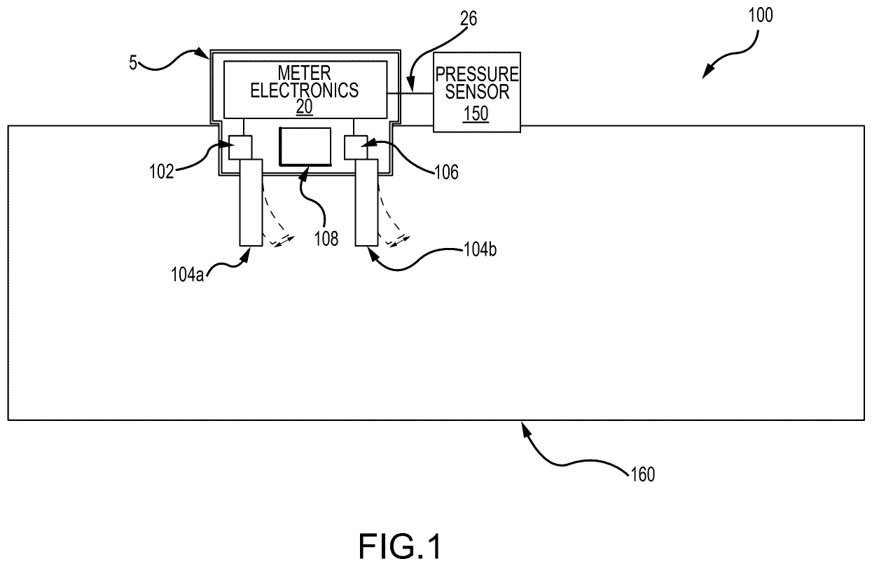

FIGS. 1-13 and the following description depict specific examples to teach those skilled in the art how to make and use the best mode of embodiments of systems and methods for determination of energy content of a flow fluid. For the purpose of teaching inventive principles, some conventional aspects have been simplified or omitted. Those skilled in the art will appreciate variations of these examples that fall within the scope of the present description. Those skilled in the art will appreciate that the features described below can be combined in various ways to form multiple variations of systems and methods for determination of energy content of a flow fluid. As a result, the embodiments described below are not limited to the specific examples described below, but only by the claims and their equivalents.

FIG. 1 shows a block diagram of an embodiment of a system 100 for determining the energy content of a flow fluid. The system may have a vibratory sensor 5, a pressure sensor 150, and a flow conduit 160. In this system, fluid is allowed to flow through the flow conduit 160 and be measured at interfaces with the vibratory sensor 5 and the pressure sensor 150. It should be appreciated that the flow fluid may be a petroleum, fuel gas, or natural gas fluid. For instance, the flow fluid may be one or more of natural gas (natural gas being a gas directly derived from a natural source), biogas, and fuel gas (fuel gas being an artificially extracted gas extracted from petroleum products).

The flow fluid may be composed of any number of substances, for instance, one or more of petroleum-based substances, alkanes, combustible substances, inert substances, oxygen, and/or the like. Petroleum-based substances may include methane, ethane, propane, propylene, isobutane, butane, and/or the like. Combustible substances may include, for instance, one or more of hydrogen, methane, ethane, propane, propylene, isobutane, butane, hydrogen sulfide, and/or the like. The inert substances may include, for instance, one or more of carbon dioxide, nitrogen, helium, carbon monoxide, water, and/or the like. The most prevalent inert substances may be carbon dioxide and nitrogen. In an embodiment, while the fluid may have some air, the fluid may be a fluid that is not entirely or majority air, such that a density of the fluid is distinct from a density of air. For instance, the fluid may be less than half air, less than a quarter air, less than a tenth air, less than none tenths air, or less than three quarters air by volume.

When determining an inferred calorific value, it may be useful to use measurements pertaining to the fluid flow, for instance, temperature (T), pressure (P), viscosity (η), and density (ρ). Any method that exists in the art is contemplated for measuring those values, and the specification merely presents examples of physical sensors and other arrangements for taking these measurements. Inferring energy content from measured parameters that are typical elements in a gas line is desirable, especially ones that do not involve significantly changing pressure (P) or temperature (T) (beyond what is necessary to take the measurements), separating discrete volumes to be tested, or combusting fluid elements. Typical measurements for a fluid flow may include, for instance, one or more of temperature (T), pressure (P), viscosity (η), and density (ρ). Implements that measure these parameters may be found on existing fluid flow lines, and, therefore, it is a significant advantage if existing elements can be used to infer energy content, for instance, a CV value inference. Energy content determinations may be made by computers 200, such as meter electronics 20, and may be made without one or more of adding components to cause temperature (T) or pressure (P) drops, without determining laminar resistances, without determining heat capacity, without determining thermal conductivity, without determining speed of sound (SOS) effects, without determining thermal diffusivity, without determining specific gravity, without determining a permittivity (dielectric constant), without determining a refractive index, and/or the like. The reason that many of these elements may be ignored when using the methods presented in this specification is that the analysis can account for the underlying effects of those other procedures and parameters.

The vibratory sensor 5 is a sensor that measures properties of the flow fluid. In various embodiments, the vibratory sensor 5 may be a Coriolis sensor, a Fork meter, a Fork densitometer, a Fork viscometer, and/or the like. The vibratory sensor 5 may be at least partially immersed into a fluid to be characterized. The fluid can comprise a liquid or a gas. Alternatively, the fluid can comprise a multi-phase fluid, such as a liquid or gas that includes entrained gas, entrained solids, multiple liquids, or combinations thereof. The vibratory sensor 5 may be mounted in a pipe or conduit, a tank, a container, or other fluid vessels. The vibratory sensor 5 can also be mounted in a manifold or similar structure for directing a fluid flow. However, other mounting arrangements are contemplated and are within the scope of the description and claims.

The vibratory sensor 5 may have a meter electronics 20, a driver 102, a first tine 104a, a second tine 104b, a response sensor 106, a temperature sensor 108, and a communication link 26. The vibratory sensor 5 operates to provide fluid measurements. The vibratory sensor 5 may provide fluid measurements including, for instance, one or more of a fluid density (ρ), fluid temperature (T), a fluid viscosity (η), a mass flowrate, a volumetric flowrate, and a pressure (P) for a fluid, including flowing or non-flowing fluids. This listing is not exhaustive and the vibratory sensor 5 may measure or determine other fluid characteristics.

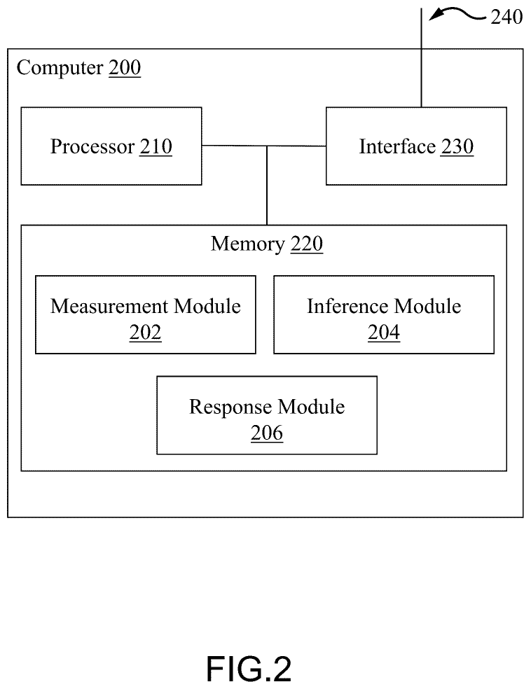

The meter electronics 20 is a processing circuit that processes raw signal data for taking measurements and/or processing programming modules. The meter electronics 20 may be an embodiment of the computer 200 shown in FIG. 2. The meter electronics 20 controls operation of the driver 102 and the response sensor 106 of the vibratory sensor 5 and can provide electrical power to the driver 102 and the response sensor 106. For example, the meter electronics 20 may generate a drive signal and provide the generated drive signal to the driver 102 to generate vibrations in the first tine 104a. The generated drive signal can control the vibrational amplitude and frequency of the first tine 104a. The generated drive signal can also control the vibrational duration and/or vibrational timing.

The driver 102 is an element that drives motions. The first tine 104a is an element that is vibrated and interacts with a fluid. The driver 102 may receive drive signals from the meter electronics 20 to vibrate the first tine 104a. The second tine 104b is another immersed element that has a resulting vibration out of phase with the vibration of the first tine 104a. The second tine 104b is coupled to a response sensor that measures the vibratory response of the second tine 104b, such that the relationship between the vibratory response of the second tine 104b and the driver signal applied to the driver 102 that drives the first tine 104a, is representative of properties of the fluid. These vibrations may be driven to allow for flow fluid and/or fluid flow measurements to be determined by the meter electronics 20. The temperature sensor 108 is a device that measures temperature. Fluid and/or fluid flow measurements may have temperature dependencies, so the temperature sensor 108 may provide temperature data to the meter electronics 20 for use in the measurements.

The meter electronics 20 can receive a vibration signal or signals from a response sensor 106 that detects motion and/or vibrations of the second tine 104b. In an embodiment, the meter electronics 20 may drive the vibratory element in a phase lock, such that the command signal provided to the driver 102 and the response signal received from the response sensor 106 are phase locked. The meter electronics 20 may process the vibration signal or signals to generate a density (ρ) measurement, for example. The meter electronics 20 processes the vibration signal or signals received from the response sensor 106 to determine a frequency of the signal or signals. Further, or in addition, the meter electronics 20 processes the vibration signal or signals to determine other characteristics of the fluid, such as a viscosity (η). The meter electronics may also determine a phase difference between signals, that can be processed to determine a fluid flow rate, for example. As can be appreciated, the phase difference is typically measured or expressed in spatial units such as degrees or radians although any suitable unit can be employed such as time-based units. If time-based units are employed, then the phase difference may be referred to by those in the art as a time delay between the vibration signal and the drive signal. Other vibrational response characteristics and/or fluid measurements are contemplated and are within the scope of the description and claims.

The meter electronics 20 can be further coupled to a communication link 26. The meter electronics 20 may communicate the vibration signal over the communication link 26. The meter electronics 20 may also process the received vibration signal to generate a measurement value or values and may communicate the measurement value or values over a communication link 26. In addition, the meter electronics 20 can receive information over the communication link 26. For example, the meter electronics 20 may receive commands, updates, operational values or operational value changes, and/or programming updates or changes over the communication link 26.

The vibratory sensor 5 may provide a drive signal for the driver using a closed-loop circuit. The drive signal is typically based on the received vibration signal. The closed-loop circuit may modify or incorporate the vibration signal or parameters of the vibration signal into the drive signal. For example, the drive signal may be an amplified, modulated, or an otherwise modified version of the received vibration signal. The received vibration signal can therefore comprise a feedback that enables the closed-loop circuit to achieve a target frequency or phase difference. Using the feedback, the closed-loop circuit incrementally changes the drive frequency and monitors the vibration signal until the target phase is reached, such that the drive frequency and vibration signal are phase locked at or near the target phase.

Fluid properties, such as the viscosity (η) and density (ρ) of the fluid, can be determined from the frequencies where the phase difference between the drive signal and the vibration signal is 135° and 45°. These desired phase differences, denoted as first off-resonant phase difference ϕ1 and second off-resonant phase difference ϕ2, can correspond to the half power or 3 dB frequencies. The first off-resonant frequency ω1 is defined as a frequency where the first off-resonant phase difference ϕ1 is 135°. The second off-resonant frequency ω2 is defined as a frequency where the second off-resonant phase difference ϕ2 is 45°. Density (ρ) measurements made at the second off-resonant frequency ω2 can be independent of fluid viscosity (η). Accordingly, density (ρ) measurements made where the second off-resonant phase difference ϕ2 is 45° can be more accurate than density (ρ) measurements made at other phase differences.

In some embodiments, the vibratory sensor 5 may only determine one of the density (ρ) and viscosity (η) with another implement determining the other of the density (ρ) and viscosity (η), the other implement perhaps being a different vibratory meter.

In one embodiment, the vibratory sensor 5 may have an inert sensor that measures a percent composition of an inert substance, for instance, a percent composition by volume of carbon dioxide (% CO₂). In another embodiment, the system may receive % CO2 values from a different apparatus. The % CO₂ may be used in an inferential relationship with the other parameters discussed.

In alternative embodiments, the vibratory sensor 5 may be different from the vibratory sensor 5 shown in FIG. 1. For instance, in other embodiments, the vibratory sensor 5 may not be a fork meter with tines. In alternative embodiments, the vibratory sensor 5 may be a gas density meter which has a vibrating cylinder rather than tines. Any vibratory sensor 5 that can determine one or more of density (ρ) and viscosity (η) may be used.

The pressure sensor 150 is a sensor that determines the pressure (P) of a flow fluid. Examples of the pressure sensor 150 may include, for instance, piezoelectric sensors, strain gages, and/or the like. The pressure sensor 150 may be configured to transmit data representing pressure (P) measurements or raw data to be used in determining pressure (P) measurements to the vibratory sensor 5, perhaps through the datalink 26 of the meter electronics 20. In an embodiment, the pressure sensor 150 is in very close proximity to the vibratory sensor 5, in order to ensure that the measurements of temperature (T), pressure (P), density (ρ) and viscosity (η) are related to a single portion of the flow fluid at the time of measurement, such that the temperature (T), pressure (P), density (ρ), and viscosity (η) for particular portions of the fluid flow are measured essentially at the same time. The pressure sensor 150 may be communicatively coupled to one or more of the vibratory sensor 5 and/or the meter electronics 20 via one or more of the communication link 26 and/or the interface 230. In one embodiment, the pressure sensor 150 may be integrated into the vibratory meter, such that any measurements and determinations can be processed by the meter electronics 20.

The conduit 160 is a fluid flow conduit. The vibratory sensor 5 and/or the pressure sensor 150 may be embedded in or attached to the surface of the conduit 160 or may have conduit elements to be connected in series with the conduit 160, in order to allow fluid flowing in the conduit 160 to interact with elements of the vibratory sensor 5 and/or the pressure sensor 150. In an embodiment, the conduit 160 may be a bypass line or side channel from a different conduit, perhaps allowing the measurements to affect the fluid flow less than if the vibratory sensor 5 and the pressure sensor 150 interacted with the fluid flowing in the different conduit.

FIG. 2 shows a block diagram of an embodiment of a computer 200. In an embodiment, the computer 200 may be a meter electronics, for instance, the meter electronics 20. In various embodiments the computer 200 may be comprised of application specific integrated circuits or may have a discrete processor and memory elements, the processor elements for processing commands from and storing data on the memory elements. The computer 200 may be an isolated physical system, a virtual machine, and/or may be established in a cloud computing environment. The computer 200 may be configured to accomplish any method steps presented in this description, for instance, any of the procedures and the capabilities of the inference module 204, and any steps in the specification for determining and/or using an inferential relationship, for instance, determining and/or using coefficient constants.

The computer system may have a processor 210, a memory 220, an interface 230, and a communicative coupler 240. The memory 220 may store and/or may have integrated circuits representing, for instance, a measurement module 202, an inference module 204, and a response module 206. In various embodiments, the computer system 200 may have other computer elements integrated into the stated elements or in addition to or in communication with the stated computer elements, for instance, buses, other communication protocols, and the like.

The processor 210 is a data processing element. The processor 210 may be any element used for processing such as a central processing unit, application specific integrated circuit, other integrated circuit, an analog controller, graphics processing unit, field programmable gate array, any combination of these or other common processing elements and/or the like. The processor 210 may have cache memory to store processing data. The processor 210 may benefit from the methods in this specification, as the methods may enhance the resolution of calculations and reduce error of those calculations using the inventive procedures presented.

The memory 220 is a device for electronic storage. The memory 220 may be any non-transitory storage medium and may include one, some, or all of a hard drive, solid state drive, volatile memory, integrated circuits, a field programmable gate array, random access memory, read-only memory, dynamic random-access memory, erasable programmable read-only memory, electrically erasable programmable read-only memory, cache memory and/or the like. The processor 210 may execute commands from and utilize data stored in the memory 220.

The computer system 200 may be configured to store any data that will be used by the measurement module 202, the inference module 204, and/or the response module 206 and may store historical data for any amount of time representing any parameter received or used by the measurement module 202, the inference module 204, and/or the response module 206 in the memory 220, perhaps with time stamps representing when the data was taken or determined. The computer system 200 may also store any data that represents determinations of any intermediates in the memory 220. While the measurement module 202, the inference module 204, and/or the response module 206 are displayed as three separate and discrete modules, the specification contemplates any number (even one or the three as specified) and variety of modules working in concert to accomplish the methods expressed in this specification.

The measurement module 202 is a module used to receive data and determine flow measurements. Fluid measurements may include one or more of a density (ρ), a temperature (T), a pressure (P), and a viscosity (η). The measurement module 202 may determine drive frequencies and receive data responses to be processed into relevant measurements. In some embodiments, the measurement module 202 may receive data from elements of the flow sensor 5 and/or the pressure sensor 150. The data may come in the form of raw signal data and/or derivative measurements. For instance, data may be received from the pressure sensor 150 representing raw data or pressure (P) values determined by the pressure sensor 150. These measurements may be transmitted to the inference module 204 in order to make inferential determinations, perhaps inferential determinations of energy content.

The inference module 204 is a module used to make inferential determinations from measured values. The inference module 204 may be used to make any number of inferential determinations including inferred energy content of a flow fluid. The energy content may be represented by any metric, for instance, CV and Wobbe index. For the purposes of disclosure in this specification, CV and energy content may be used interchangeably, and, when a determination of CV is mentioned, embodiments of other energy content metrics are contemplated. However, in the claims, if calorific value (CV) is specified, it only refers specifically to calorific value (CV) and not other energy content metrics.

In an embodiment, the inference module 204 may determine parameters for determining an inferential relationship by determining coefficient constants for coefficients used in terms of the inferential relationship. The inference module 204 may be such that it uses preestablished data to determine elements of the inferential relationship, and/or the inference module 204 may use preestablished or predetermined parameters to infer CVs from live measurements. The predetermined parameters may include the coefficient constants. The coefficient constants may be for a particular fluid or for a particular class of fluids, for instance, one or more of fuel gas, natural gas, flare gas, liquefied natural gas, biogas, and a class of fluid associated with a geographic region. In an embodiment, the inference module 204 may only determine the parameters of the inferential relationship to be used later or by another device to make live inferred CV inferences. In another embodiment, the inference module 204 may simply use predetermined coefficient constants to generate live inferred CVs of a fluid flow. In still another embodiment, the inference module 204 may both determine the predetermined coefficient constants from preexisting data and apply the predetermined coefficient constants to live measurements to make live inferences of inferred CV.

The inference module 204 may make CV inferences by accounting for temperature (T) and pressure (P) dependence of parameters and their relationships with CV. For instance, the CV may be determined by accounting for the temperature (T) and/or pressure (P) dependence of density (ρ) and viscosity (η). Density (ρ) and viscosity (η) may be adjusted, by one or more of the measurement module 202 and the inference module 204, to account for temperature (T) and pressure (P) effects. The inference module 204 may express a relationship between CV and one or more of density (ρ) and viscosity (η) as a relationship between CV and inferential relationship terms. The inferential relationship terms may include one or more of a shift term (A), density term (B), and/or viscosity term (C). The relationship between the inferred CV and the inferential relationship terms may be that the CV is inferred as a sum of one or more of the inferential relationship terms. The inference module 204 may infer CV values by adjusting parameters associated with the density (ρ) and viscosity (η) measurement values, for instance, by establishing temperature (T) and pressure (P) dependent coefficients for one or more of at least one density term (B), at least one viscosity term (C), and/or at least one shift term (A) of a CV determination. The inference module 204 may determine and/or store coefficient constants (e.g. a₁-a₄, b₁-b₄, c₁-c₄, and/or d₁-d₄) used to determine the temperature (T) and/or pressure (P) dependent coefficients. These coefficient constants may be dependent upon certain parameters, for instance, one or more of, the substances in the fluid flow, a class of the substances in the fluid flow, and/or the like. The coefficient constants (e.g. a₁-a₄, b₁-b₄, c₁-c₄, and/or d₁-d₄) may be determined using a number of different mixture compositions over different temperature and pressure conditions using analytic techniques, for instance, regression or probabilistic or statistical methods. The coefficient constants (e.g. a₁-a₄, b₁-b₄, c₁-c₄, and/or d₁-d₄) may be determined substantially simultaneously using these techniques.

In an embodiment, the coefficient constants may already be determined based on the parameters, for instance, known elements of the flow or the classes of fluids to which the coefficient constants correspond. In an embodiment in which the coefficient constants have already been determined, they may be referred to as predetermined coefficient constants. The inference module 204 may infer an inferred energy content using the predetermined coefficient constants, perhaps using predetermined coefficient constants associated with the fluid or a class of fluids of which the fluid is a member. Classes of substances may include one or more of fuel gas, natural gas, flare gas, liquefied natural gas, shale gas, biogas, and a class of fluid associated with a geographic region. Geographic regions may include, for instance, a particular continent, a particular country, a particular city, a particular county, and/or the like. For instance, the class of fluids may be natural gas. In another embodiment, the class of fluids may be natural gas from a region such as North America.

The inference module 204 may use relationships between one or more of temperature (T), pressure (P), density (ρ), and viscosity (η) values. One or more of temperature (T), pressure (P), density (ρ), and viscosity (η) values may be measured values, for instance, measured by one or more of the vibratory sensor 5 or the pressure sensor 150 show in FIG. 1. In various embodiments, the inference module 204 may be able to assume certain parameter values because process controls allow for consistency in one or more of the temperature (T), pressure (P), density (ρ), and viscosity (η) values. For instance, in an embodiment, one or more of the pressure (P) and the temperature (T) may be determined to be sufficiently consistent that a constant value for the one or more of the pressure (P) and the temperature (T) may be used instead of a measurement.

The inference module 204 may infer a CV value by accounting for and/or determining one or more of a density term (B), a viscosity term (C), and/or a shift term (A). In an embodiment, an inferential relationship to infer CV may be:

$$CV = A + B + C \qquad ( 2 )$$

The density term (B) accounts for the density (ρ) effects of the flow fluid on CV inferences. The density term (B) may have an inverse relationship with CV, such that an increase in the density term (B) and/or the density (ρ) one or more of reduces the effect the density (ρ) has on the CV and decreases the CV inferred from the increased density (ρ). The density term (B) may not account for the density of pure or environmental air (ρₐᵢᵣ) separately of the fluid. Also, the measured density (ρ) may not be a measurement of the density of pure air (ρₐᵢᵣ). Also, the inferential relationship may not account for a measurement of the density of pure or environmental air (ρₐᵢᵣ). The density term (B) may have a temperature (T) and/or pressure (P) dependent density term coefficient (k₂(P,T)) that can be determined and/or applied dynamically, perhaps using derived or predetermined constants, for instance, density coefficient constants (b₁-b₄). The density term (B) may be separate from the viscosity term (C) such that the density term (B) does not account for a viscosity (η) or a relationship between viscosity (η) and density (ρ). Further, the density term (B) may be such that density (ρ) is neither multiplied nor divided by any quantity associated with viscosity (η). In an embodiment, the lack of dependence of the density term (B) on viscosity (η) may be expressed as the temperature (T) and/or pressure (P) dependent density term coefficient (k₂(P,T)) and may be applied to the density (ρ) such that the inferential relationship (or, perhaps, equation) may be expressed without having the temperature (T) and/or pressure (P) dependent density term coefficient (k₂(P,T)) multiplied or divided by viscosity (η). In an embodiment, the density term (B) may be:

$$B = {k}_{2} ( P , T ) \times \frac{1}{ρ} \qquad ( 3 )$$

In an embodiment, the temperature (T) and/or pressure (P) dependent density term coefficient (k₂(P,T)) may be expressed as a temperature dependent relationship with density coefficient constants (b₁-b₄). In an embodiment, the temperature (T) and/or pressure (P) dependent density term coefficient (k₂(P,T)) may be expressed as:

$${k}_{2} ( P , T ) = [ {b}_{1} + {b}_{2} ( T - 20 ) ] + [ {b}_{3} + {b}_{4} ( T - 20 ) ] \times P \qquad ( 4 )$$

The viscosity term (C) accounts for the viscosity (η) effects of the flow fluid on CV inferences. The viscosity term (C) may have a direct relationship with CV, such that an increase in viscosity (η) and/or the viscosity term (C) one or more of increases the effect the viscosity (η) has on the CV and increases the CV inferred from the increased viscosity (η). The viscosity term (C) may have a temperature (T) and/or pressure (P) dependent viscosity term coefficient (k₃(P,T)) that can be determined dynamically, perhaps using derived or predetermined constants, for instance, viscosity coefficient constants (c₁-c₄). In an embodiment, the viscosity term (C) may be:

$$C = {k}_{3} ( P , T ) \times η \qquad ( 5 )$$

In an embodiment, the temperature (T) and/or pressure (P) dependent viscosity term coefficient (k₃(P,T)) may be expressed as a temperature dependent relationship with viscosity coefficient constants (c1-c4). In an embodiment, the temperature (T) and/or pressure (P) dependent viscosity term coefficient (k₃(P,T)) may be expressed as:

$${k}_{3} ( P , T ) = [ {c}_{1} + {c}_{2} ( T - 20 ) ] + [ {c}_{3} + {c}_{4} ( T - 20 ) ] \times P \qquad ( 6 )$$

The shift term (A) is a term that is a baseline value for CV. The shift term (A) may be temperature (T) and/or pressure (P) dependent. The shift term may be directly related to the CV determined based on an inferential relationship. The shift term (A) may include, for instance, a temperature (T) and/or pressure (P) dependent shift term coefficient (k₁(P,T)). In different embodiments, the shift term (A) may be only a coefficient (essentially, multiplied by a value of 1) or may be applied to other measurement parameters. In an embodiment, the shift term (A) may be:

$$A = {k}_{1} ( P , T ) \qquad ( 7 )$$

In an embodiment, the temperature (T) and/or pressure (P) dependent shift term coefficient (k₁(P,T)) may be expressed as a temperature dependent relationship with shift coefficient constants (a1-a4). In an embodiment, the temperature (T) and/or pressure (P) dependent shift term coefficient (k₁(P,T)) may be expressed as:

$${k}_{1} ( P , T ) = [ {a}_{1} + {a}_{2} ( T - 20 ) ] + [ {a}_{3} + {a}_{4} ( T - 20 ) ] \times P \qquad ( 8 )$$

The inference module 204 may use any of the relationships expressed in Eqs. (2)-(8) to inferentially determine a CV value. In an embodiment, Eqs. (2), (3), (5), and (7) may be combined to form an embodiment of an inferential relationship between CV and both of density and viscosity, the embodiment perhaps being:

$$CV = {k}_{1} ( P , T ) + {k}_{2} ( P , T ) \times \frac{1}{ρ} + {k}_{3} ( P , T ) \times η \qquad ( 9 )$$

In an embodiment, the temperature (T) and/or pressure (P) dependent coefficients k₁(P,T), k₂(P,T), and k₃(P,T) may be determined or used to infer energy content using relationships expressed in Eqs. (8), (4), and (6), respectively.

In an embodiment, the inference module 204 may determine coefficient constants by using known values and/or relationships of flow fluids and performing regression analysis. For instance, the coefficient constants may be determined by performing regression analysis based on known values of one or more flow fluids. Known values may include measured CV values at particular dependency parameters, the dependency parameters perhaps including one or more of, for instance, the identity and/or class of the fluid, the relative composition (of different substances) of the fluid, a temperature (T), a pressure (P), a density (ρ), and/or a viscosity (η). The dependency parameters may be expressed as relationships or equations that relate the dependency parameters instead of or in addition to discrete values of the dependency parameters. In an embodiment, a number of different measured CV values and corresponding dependency parameters may be classified by classes of fluid, such that the properties of substances in the class of fluid may have sufficiently similar properties to apply one set of coefficient constants to all of the fluids in the class. For instance, classes of fluids may include one or more of fuel gas, natural gas, flare gas, liquefied natural gas, and/or biogas. The inference module 204 may run regression analysis on CV values and corresponding dependency parameters on one or more of the substances characterized in a class in order to determine class-specific coefficient constants. In an embodiment, the inference module 204 may store predetermined class-specific and/or substance specific coefficient constants for use during periods when CV is to be inferred.

In an embodiment, the inference module 204 may determine the coefficient constants. In another embodiment, the inference module 204 may use predetermined coefficient constants, perhaps identified by expected fluid flow substances or by class. The inference module 204 may determine class-specific coefficient constants and/or the inference module 204 may use predetermined class-specific coefficient constants.

In an embodiment of an operation, the inference module 204 may use the coefficient constants to determine an inferred CV of a flow fluid. For instance, the CV may be determined inferentially by applying at least one relationship between coefficient constants and at least one of a measured temperature (T), measured pressure (P), measured density (ρ), and a measured viscosity (η), for instance, one or more of the relationships expressed in Eqs. (2)-(13). In other embodiments, one or more of the pressure and temperature may be assumed or assumed to be within a range instead of being measured. In various embodiments, the inference module 204 may be configured to infer CV values without one or more of adding components to effect temperature (T) or pressure (P) drops, determining laminar resistances, determining heat capacity, determining thermal conductivity, determining thermal diffusivity, determining specific gravity, and/or the like. The inference module 124 may also evaluate a density term (B) without consideration of viscosity (η) and/or without multiplying or dividing a density (ρ) by a viscosity (η) in the density term (B).

In another embodiment, a further term may be incorporated into the inferential relationship. As can be seen in the AGA 5 equation, the inert concentration can have a significant effect. In an embodiment, the system may be further configured to measure the percentage composition (perhaps by volume or mass) of certain inert substances, for instance, one or more of carbon dioxide (% CO₂) and nitrogen (% N₂). In this embodiment, Eqs. (2) and (13) may be augmented by a further sum of an inert term (D). The inert term (D) may have a temperature (T) and pressure (P) dependent coefficient (k₄(P,T)) determined from inert term coefficient constants (d₁-d₄). The inert term (D) may take the form of Eq. (10).

$$D = {k}_{4} ( P , T ) \times \% {CO}_{2} \qquad ( 10 )$$

For instance, the inferential relationship may take the form of Eq. (11).

$$CV = A + B + C + D \qquad ( 11 )$$

In an embodiment, Eq. (11) can be expressed as Eq. (12).

$$CV = {k}_{1} ( P , T ) + {k}_{2} ( P , T ) \times \frac{1}{ρ} + {k}_{3} ( P , T ) \times η + {k}_{4} ( P , T ) \times \% {CO}_{2} \qquad ( 12 )$$

An example of an expression to determine the inert term coefficient (k₄(P,T)) is Eq. 13.

$${k}_{4} ( P , T ) = [ {d}_{1} + {d}_{2} ( T - 20 ) ] + [ {d}_{3} + {d}_{4} ( T - 20 ) ] \times P \qquad ( 13 )$$

The terms (A, B, C, and/or D) may be determined in any order or may be determined substantially simultaneously, using analytic techniques, for instance, regression or probabilistic or statistical methods.

The response module 206 is a module that takes actions responsive to determinations and operations of the measurement module 202 and/or the inference module 204. For instance, in response to a determination of an inferential CV, the response module 206 may transmit data representing the CV to or store data representing the CV in the memory 220. The response module 206 may transmit data representing the CV to external components, for instance, a display or other compute device. Other responsive actions that may be taken by the response module 206 or by compute devices to which the response module 206 transmits an inferred CV may include one or more of, for instance, determining a price based on the inferred CV, regulating gas in a distribution (e.g. adding propane if the CV value is too low), and/or regulating a burner control. Other responsive actions known in the art may be taken by the response module 206 and are contemplated by this specification.

The capabilities of the measurement module 202, the inference module 204, and/or the response module 206 are contemplated and reflect the methods that are performed in the flowcharts presented. All methods in this specification are contemplated with respect to each flowchart and orders specified or, when it is specified that the order does not matter, inform the flowcharts, but all methods and capabilities of the measurement module 202, the inference module 204, and/or the response module 206 are contemplated for the purposes of any steps in flowcharts and/or method claims that follow.

The interface 230 is an input/output device that allows communication between the computer 200 and external elements. The interface 230 is capable of connecting the computer system 200 to external elements using known technologies, for instance, universal serial bus, serial communication, serial advanced technology attachments, and/or the like. The interface 230 may have a communicative coupler 240. In an embodiment the communicative coupler 240 may be the communication link 26 or may be communicatively coupled to the communication link 26. External elements to which the interface 230 may be coupled include one or more of the driver 102, the response sensor 106, the temperature sensor 108, and/or an external compute device.

### Flowcharts

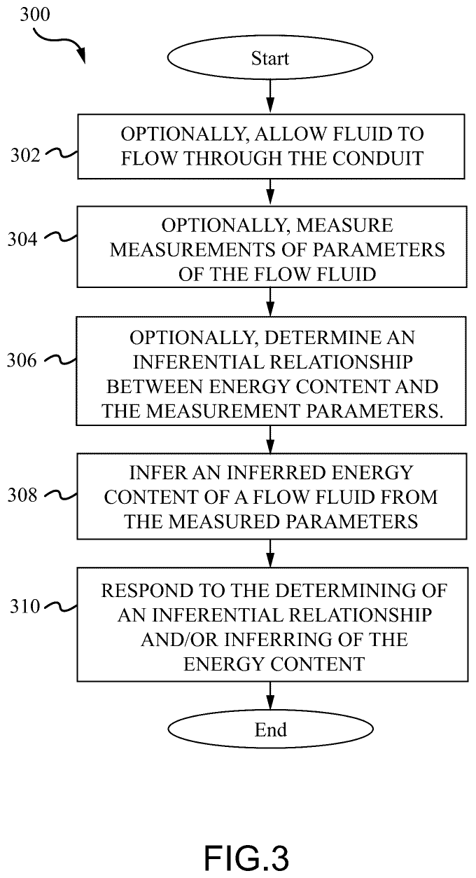

FIGS. 3-9 show flowcharts of embodiments of methods for determining and/or using inferential relationships to infer live inferred CV values of a flow fluid. The methods disclosed in the flowcharts are non-exhaustive and merely demonstrate potential embodiments of steps and orders. The methods are contemplated in the context of the entire specification, including elements disclosed in descriptions of vibratory sensor 5, pressure sensor 150, meter electronics 20, the computer 200, the measurement module 202, the inference module 204, and/or the response module 206 disclosed in FIGS. 1 and 2.

FIG. 3 shows a flowchart of an embodiment of a method 300 for using an inferential relationship between measured parameters and fluid energy content. The vibratory sensor 5, pressure sensor 150, conduit 160, measurement module 202, inference module 204, and response module 206 referred to in method 300 may be the vibratory sensor 5, pressure sensor 150, conduit 160, measurement module 202, inference module 204, and response module 206 as disclosed in FIGS. 1 and 2, although any suitable alternatives may be employed in alternative embodiments. All methods for accomplishing these steps disclosed in this specification are contemplated, including all of the capabilities of the vibratory sensor 5, pressure sensor 150, conduit 160, measurement module 202, inference module 204, and response module 206.

Step 302 is optionally, allowing fluid to flow through the conduit 160. The fluid may flow through the conduit 160 such that the flow fluid of the fluid flow interacts with the vibratory sensor 5 and/or the pressure sensor 150.

Step 304 is optionally, measuring, by the measurement module 202 measurements of parameters of the flow fluid. These measurement parameters may be used, perhaps by the inference module 204, in determining and/or using an inferential relationship to determine an energy content of a flow fluid from the measurement parameters. The measurement parameters may include one or more of a temperature (T), a pressure (P), a density (ρ), and/or a viscosity (η). In an embodiment, the measurement parameters may be received from another process or input, making the parameters predetermined parameters. These predetermined measurement parameters may be used with predetermined energy content values determined in a controlled environment to establish relationships between gas content and the predetermined measurement parameters. In another embodiment, the measurement module 202 may determine the measurement parameters, perhaps by receiving inputs from the meter electronics 20 of the vibratory sensor 5. These measurement parameters may have been determined by the meter electronics 20 and/or the pressure sensor 150 from methods stated with respect to taking measurements of a flow fluid using the vibratory sensor 5.

Step 306 is optionally, determining, by the inference module 204, an inferential relationship between energy content and the measurement parameters. The inference module 204 may determine inferential relationships between measured parameters and energy content using any and all capabilities of the inference module 204 stated in this specification. For instance, the inference module 204 may use existing data correlated to known energy content values in order to conduct a regression to determine the inferential relationship, perhaps using elements of the relationships expressed by Eqs. (2)-(13).

Step 308 is inferring, by the inference module 204, an inferred energy content of a flow fluid from the measured parameters. The inference module 204 may use any and all relationships and procedures expressed as capabilities of the inference module 204 in order to infer energy content from the measured parameters. For instance, the inference module may use relationships expressed by Eqs. (2)-(13) to infer energy content of the flow fluid. The inference module 204 may use the measurements of parameters in step 304 (or provided from another source) and may use relationships and corresponding parameters in step 306 (or provided from another source) to determine the inferred energy content.

Step 310 is optionally, responding, by the response module 206, to the determining of an inferential relationship and/or inferring of the energy content. Any response by the response module 206 expressed in this specification is contemplated. For instance, the response module 206 may respond by one or more of storing or transmitting parameters, storing or transmitting relationships between parameters, storing or transmitting coefficient constants, storing or transmitting inferred energy content values, determining a price based on the inferred CV, storing or transmitting a price based on the inferred CV, regulating gas in a distribution network (e.g. adding propane if the CV value is too low), and/or regulating a burner control.

In an embodiment, each of the steps of the method shown in FIG. 3 is a distinct step. In another embodiment, although depicted as distinct steps in FIG. 3, steps 302-310 may not be distinct steps. In other embodiments, the method shown in FIG. 3 may not have all of the above steps and/or may have other steps in addition to or instead of those listed above. The steps of the method shown in FIG. 3 may be performed in another order. Subsets of the steps listed above as part of the method shown in FIG. 3 may be used to form their own method. The steps of method 300 may be repeated in any combination and order any number of times, for instance, continuously looping in order to provide consistent energy content values.

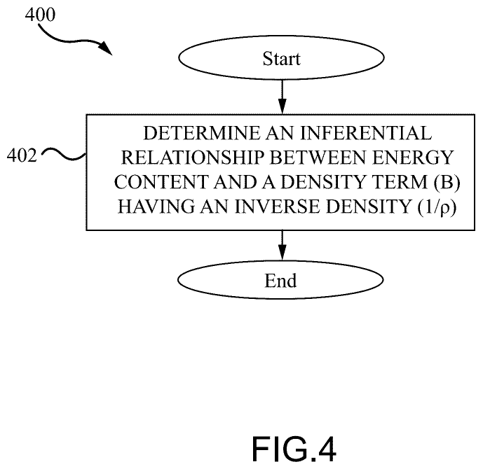

FIG. 4 shows a flowchart of an embodiment of a method 400 for determining an inferential relationship between measured parameters and flow fluid energy content. The vibratory sensor 5, pressure sensor 150, conduit 160, measurement module 202, inference module 204, and response module 206 referred to in method 300 may be the vibratory sensor 5, pressure sensor 150, conduit 160, measurement module 202, inference module 204, and response module 206 as disclosed in FIGS. 1 and 2, although any suitable alternatives may be employed in alternative embodiments. All methods for accomplishing these steps disclosed in this specification are contemplated, including all of the capabilities of the vibratory sensor 5, pressure sensor 150, conduit 160, measurement module 202, inference module 204, and response module 206. In an embodiment, the steps of method 400 may be conducted by a computer 200 that receives preexisting data, without the need to take any measurements by elements used in method 400.

Step 402 is determining, by the inference module 204, an inferential relationship between energy content and a density term (B) having an inverse density (1/ρ). The relationship may yield an inferred energy content. The use of an inverse density (1/ρ) in a density term (B) may provide more consistent results. The density term (B) may be such that it can be expressed without having any relationship to viscosity (η). The density term (B) may be such that it can be expressed without having any relationship to specific gravity. The density term (B) may not account for the density of pure or environmental air (ρₐᵢᵣ) separately of the fluid. Also, the measured density (ρ) may not be a measurement of the density of pure air (ρₐᵢᵣ). Also, the inferential relationship may not account for a measurement of the density of pure or environmental air (ρₐᵢᵣ). The inference module 204 may use any capabilities of the inference module 204 expressed in this specification to accomplish the determining of the inferential relationship between energy content and the density term (B) having an inverse density. The determination of the relationship may yield coefficient constants that characterize the relationship and can be used as predetermined coefficient constants in a live energy content determination, for instance, by inputting the predetermined coefficient constants into the relationship that relates live measurements to generate an inferred live energy content value. Step 402 may be an embodiment of step 306.

In other embodiments, the method shown in FIG. 4 may have other steps in addition to or instead of the step listed above. Substeps of the step listed above as part of the method shown in FIG. 4 may be used to form their own method. The step of method 400 may be repeated any number of times, for instance, to determine energy content for different flow fluids and/or classes of flow fluids.

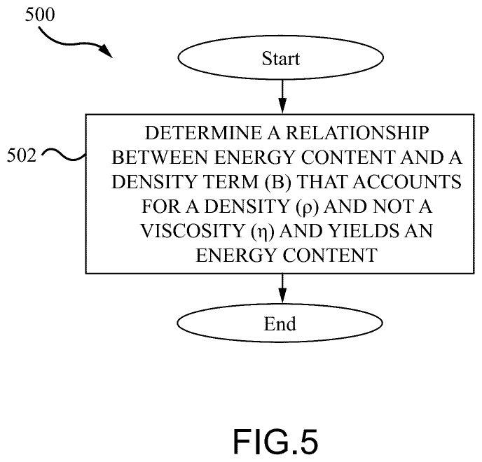

FIG. 5 shows a flowchart of another embodiment of a method 500 for determining an inferential relationship between measured parameters and flow fluid energy content. The vibratory sensor 5, pressure sensor 150, conduit 160, measurement module 202, inference module 204, and response module 206 referred to in method 300 may be the vibratory sensor 5, pressure sensor 150, conduit 160, measurement module 202, inference module 204, and response module 206 as disclosed in FIGS. 1 and 2, although any suitable measurement module 202, inference module 204, and response module 206 may be employed in alternative embodiments. All methods for accomplishing these steps disclosed in this specification are contemplated, including all of the capabilities of the vibratory sensor 5, pressure sensor 150, conduit 160, measurement module 202, inference module 204, and response module 206. In an embodiment, the steps of method 500 may be conducted by a computer 200 that receives preexisting data, without the need to take any measurements by elements used in method 500.

Step 502 is determining, by the inference module 204, a relationship between energy content and a density term (B) that accounts for a density (ρ) and not a viscosity (η) and yields an inferred energy content. The density term (B) may be such that it incorporates a density (ρ), perhaps by an inverse density (1/ρ), and can be expressed without having any relationship to viscosity (η). The density term (B) may be such that it can be expressed without having any relationship to specific gravity. The density term (B) may not account for the density of pure or environmental air (ρₐᵢᵣ) separately of the fluid. Also, the measured density (ρ) may not be a measurement of the density of pure air (ρₐᵢᵣ). Also, the inferential relationship may not account for a measurement of the density of pure or environmental air (ρₐᵢᵣ). The inference module 204 may use any capabilities of the inference module 204 expressed in this specification to accomplish the determining of the relationship between energy content and the density term (B) having an inverse density (1/ρ). The determination of the relationship may yield coefficient constants that characterize the relationship and can be used as predetermined coefficient constants in a live energy content determination, for instance, by inputting the predetermined coefficient constants into the relationship that relates live measurements to generate an inferred live energy content value. Step 502 may be an embodiment of step 306.

In other embodiments, the method shown in FIG. 5 may have other steps in addition to or instead of the step listed above. Sub steps of the step listed above as part of the method shown in FIG. 5 may be used to form their own method. The step of method 500 may be repeated any number of times, for instance, to determine energy content for different flow fluids and/or classes of flow fluids.

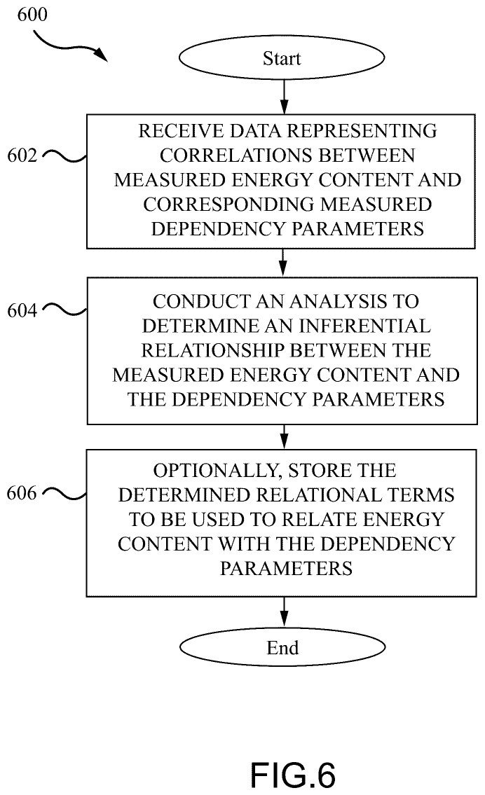

FIG. 6 shows a flowchart of still another embodiment of a method 600 for determining an inferential relationship between measured parameters and flow fluid energy content. The vibratory sensor 5, pressure sensor 150, conduit 160, measurement module 202, inference module 204, and response module 206 referred to in method 300 may be the vibratory sensor 5, pressure sensor 150, conduit 160, measurement module 202, inference module 204, and response module 206 as disclosed in FIGS. 1 and 2, although any suitable measurement module 202, inference module 204, and response module 206 may be employed in alternative embodiments. All methods for accomplishing these steps disclosed in this specification are contemplated, including all of the capabilities of the vibratory sensor 5, pressure sensor 150, conduit 160, measurement module 202, inference module 204, and response module 206. In an embodiment, the steps of method 600 may be conducted by a computer 200 that receives preexisting data, without the need to take any measurements by elements used in method 600.

Step 602 is receiving, by the inference module 204, data representing correlations between measured energy content and corresponding measured dependency parameters. The dependency parameters may include one or more of, for instance, the identity and/or class of the fluid, the relative composition (of different substances) of the fluid, a temperature (T), a pressure (P), a density (ρ), and/or a viscosity (η). All capabilities of the inference module 204 are contemplated for carrying out step 602.

Step 604 is conducting, by the inference module 204, an analysis to determine an inferential relationship between the measured energy content and the dependency parameters. The relationship may yield an inferred energy content. In an embodiment, the analysis may be a regression. The analysis may output relational terms that relate measured energy content with the dependency parameters. The determination of the relationship may yield coefficient constants that characterize the relationship and can be used as predetermined coefficient constants in a live energy content determination, for instance, by inputting the predetermined coefficient constants into the relationship that relates live measurements to generate an inferred live energy content value. All capabilities of the inference module 204 are contemplated for carrying out step 604. Steps 602 and 604, combined, may be an embodiment of step 306.

Step 606 is optionally, storing, by the inference module 204 or the response module 206, the determined relational terms to be used to relate energy content with the dependency parameters that may be determined by live measurement to yield live inferred energy content values. All capabilities of the inference module 204 and/or the response module 206 are contemplated for carrying out step 606. In an embodiment, the modules may further transmit the inferred energy content value to external devices. Step 606 may be an embodiment of step 310.

In an embodiment, each of the steps of the method shown in FIG. 6 is a distinct step. In another embodiment, although depicted as distinct steps in FIG. 6, steps 602-606 may not be distinct steps. In other embodiments, the method shown in FIG. 6 may not have all of the above steps and/or may have other steps in addition to or instead of those listed above. The steps of the method shown in FIG. 6 may be performed in another order. Subsets of the steps listed above as part of the method shown in FIG. 6 may be used to form their own method. The steps of method 600 may be repeated in any combination and order any number of times, for instance, to compute different relational values for different flow fluids and/or classes of flow fluids.

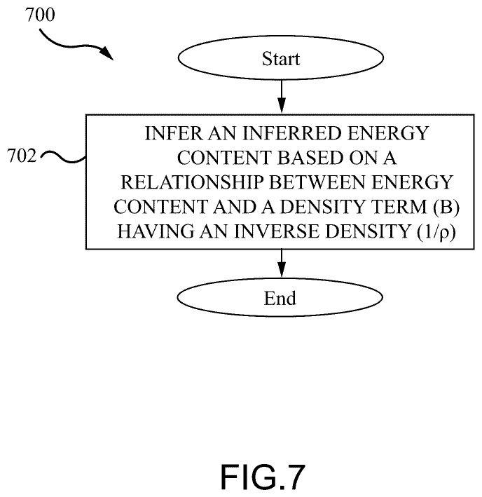

FIG. 7 shows a flowchart of an embodiment of a method 700 for inferring an energy content from measured parameters. The vibratory sensor 5, pressure sensor 150, conduit 160, measurement module 202, inference module 204, and response module 206 referred to in method 300 may be the vibratory sensor 5, pressure sensor 150, conduit 160, measurement module 202, inference module 204, and response module 206 as disclosed in FIGS. 1 and 2, although any suitable alternatives may be employed in alternative embodiments. All methods for accomplishing these steps disclosed in this specification are contemplated, including all of the capabilities of the vibratory sensor 5, pressure sensor 150, conduit 160, the measurement module 202, inference module 204, and response module 206.

Step 702 is inferring, by the inference module 204, an inferred energy content based on a relationship between energy content and a density term (B) having an inverse density (1/ρ). The relationship may yield an inferred energy content. The use of an inverse density (1/ρ) in a density term (B) may provide more consistent results. The density term (B) may be such that it can be expressed without having any relationship to viscosity. The density term (B) may not account for the density of pure or environmental air (ρₐᵢᵣ) separately of the fluid. Also, the measured density (ρ) may not be a measurement of the density of pure air (ρₐᵢᵣ). Also, the inferential relationship may not account for a measurement of the density of pure or environmental air (ρₐᵢᵣ). The inference module 204 may use any capabilities of the inference module 204 expressed in this specification to accomplish the inferring of the relationship between energy content and the density term (B) having an inverse density. The inference module may infer the inferred energy content by incorporating predetermined coefficient constants into the relationship, such that the relationship can infer an inferred energy content based on measured values and the predetermined coefficient constants. Step 702 may be an embodiment of step 308.

In other embodiments, the method shown in FIG. 7 may have other steps in addition to or instead of the step listed above. Subsets of the step listed above as part of the method shown in FIG. 7 may be used to form their own method. The step of method 700 may be repeated any number of times, for instance, to determine energy content for different flow fluids and/or classes of flow fluids.

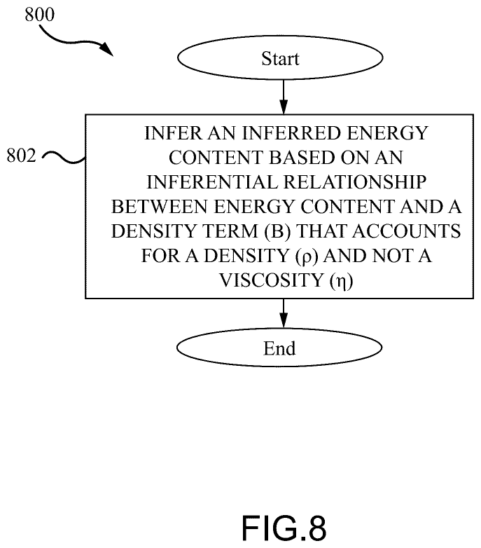

FIG. 8 shows a flowchart of another embodiment of a method 800 for inferring an energy content from measured parameters. The vibratory sensor 5, pressure sensor 150, conduit 160, measurement module 202, inference module 204, and response module 206 referred to in method 300 may be the vibratory sensor 5, pressure sensor 150, conduit 160, measurement module 202, inference module 204, and response module 206 as disclosed in FIGS. 1 and 2, although any suitable measurement module 202, inference module 204, and response module 206 may be employed in alternative embodiments. All methods for accomplishing these steps disclosed in this specification are contemplated, including all of the capabilities of the vibratory sensor 5, pressure sensor 150, conduit 160, the measurement module 202, inference module 204, and response module 206.

Step 802 is inferring, by the inference module 204, an inferred energy content based on an inferential relationship between energy content and a density term (B) that accounts for a density (ρ) and not a viscosity (η). The inferential relationship may yield an energy content. The density term (B) may be such that it incorporates a density (ρ), perhaps an inverse density (1/ρ), and can be expressed without having any relationship to viscosity (η). The density term (B) may not account for the density of pure or environmental air (ρₐᵢᵣ) separately of the fluid. Also, the measured density (ρ) may not be a measurement of the density of pure air (ρₐᵢᵣ). Also, the inferential relationship may not account for a measurement of the density of pure or environmental air (ρₐᵢᵣ). The inference module 204 may infer the inferred energy content by incorporating predetermined coefficient constants into the relationship, such that the relationship can infer an inferred energy content based on measured values and the predetermined coefficient constants. The inference module 204 may use any capabilities of the inference module 204 expressed in this specification to accomplish step 802. Step 802 may be an embodiment of step 308.

In other embodiments, the method shown in FIG. 8 may have other steps in addition to or instead of the step listed above. Subsets of the step listed above as part of the method shown in FIG. 8 may be used to form their own method. The step of method 800 may be repeated any number of times, for instance, to determine energy content for different flow fluids and/or classes of flow fluids.

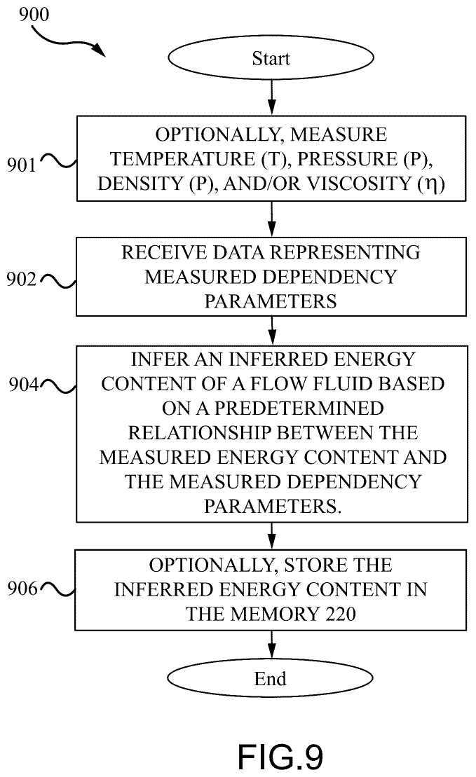

FIG. 9 shows a flowchart of still another embodiment of a method 900 for inferring an energy content from measured parameters. The vibratory sensor 5, pressure sensor 150, conduit 160, measurement module 202, inference module 204, and response module 206 referred to in method 300 may be the vibratory sensor 5, pressure sensor 150, conduit 160, measurement module 202, inference module 204, and response module 206 as disclosed in FIGS. 1 and 2, although any suitable alternatives may be employed in alternative embodiments. All methods for accomplishing these steps disclosed in this specification are contemplated, including all of the capabilities of the vibratory sensor 5, pressure sensor 150, conduit 160, measurement module 202, inference module 204, and response module 206. In an embodiment, the steps of method 900 may be conducted by a computer 200 that receives preexisting data, without the need to take any measurements by elements used in method 900.

Step 901 is optionally, measuring, by one or more of the vibratory sensor 5 and the pressure sensor 150, a temperature (T), a pressure (P), a density (ρ), and/or a viscosity (η). Step 901 may be an embodiment of step 304. The measuring step is one in which fluid is allowed to interact with one or more of the vibratory sensor 5 and the pressure sensor 150. In an embodiment, the fluid is introduced to a conduit 160 and the fluid flows to interact with the one or more of the vibratory sensor 5 and the pressure sensor 150. In an embodiment, the vibratory sensor 5 has immersed elements that may interact directly with the fluid. All methods for measuring measurable quantities of a fluid that are associated with the one or more of the vibratory sensor 5 and the pressure sensor 150, as disclosed in the specification, are contemplated to effectuate step 901.

Step 902 is receiving, by the inference module 204, data representing measured dependency parameters. The dependency parameters may include one or more of, for instance, the identity and/or class of the fluid, an expected or estimated relative composition (of different substances) of the fluid, a temperature (T), a pressure (P), a density (ρ), and/or a viscosity (η). In various embodiments, a vibratory sensor 5 may be used to take measurements to provide one or more of the dependency parameters. For instance, the vibratory sensor 5 may be configured to measure a temperature (T), a pressure (P), a density (ρ), and/or a viscosity (η).

In an embodiment in which a separate pressure sensor 150 is used, the pressure sensor 150 may transmit data representing the pressure (P) measurements, and, perhaps, temperature (T) measurements (or raw data representing signals received to determine pressure (P) and/or temperature (T) measurements), to the computer 200 (or perhaps the meter electronics 20 of the vibratory sensor 5) for processing. In this embodiment, one or more vibratory sensors 5 may determine density (ρ) and viscosity (η) of a flow fluid. The vibratory sensor(s) may also measure temperature (T). Measurements taken by the vibratory sensor(s) may be transmitted to computer 200 (or perhaps to internal meter electronics 20 of a vibratory sensor 5). All capabilities of the measurement module 202, computer 200, vibratory sensor 5, and pressure sensor 150 are contemplated for carrying out step 902.

In an embodiment, the user may specify the dependency parameters associated with identity of the substances, for instance, the identity and/or class of the fluid or an expected or estimated relative composition (of different substances) of the fluid. This specification may be received by the computer 200 (or meter electronics 20).

Step 904 is inferring, by the inference module 204, an inferred energy content of a flow fluid based on a predetermined relationship between the measured energy content and the measured dependency parameters. The predetermined relationship may be stored in the computer 200 (or in meter electronics 20) and may have predetermined relational terms that relate measured energy content with the dependency parameters. For instance, the inference module 204 may use predetermined coefficient constants stored in the computer 200 (or meter electronics 20) to be input into a predetermined relationship between energy content and dependency parameters to yield an inferred energy content based on one or more of the dependency parameters. In doing this, the computer 200 (or meter electronics 20) may determine energy content using measurements commonly applied to fluid flows (e.g. measurements of temperature (T), pressure (P), density (ρ), and/or viscosity (η)).

In an embodiment, the predetermined relationship is modeled by Eqs. (2)-(13). All capabilities of the inference module 204 are contemplated for carrying out step 904. Steps 902 and 904, in combination, may be an embodiment of step 308.

Step 906 is optionally, storing, by the inference module 204 or the response module 206, the inferred energy content in the memory 220. All capabilities of the inference module 204 and/or the response module 206 are contemplated for carrying out step 906. Step 906 may be an embodiment of step 310.

In an embodiment, each of the steps of the method shown in FIG. 9 is a distinct step. In another embodiment, although depicted as distinct steps in FIG. 9, steps 902-906 may not be distinct steps. In other embodiments, the method shown in FIG. 9 may not have all of the above steps and/or may have other steps in addition to or instead of those listed above. The steps of the method shown in FIG. 9 may be performed in another order. Subsets of the steps listed above as part of the method shown in FIG. 9 may be used to form their own method. The steps of method 900 may be repeated in any combination and order any number of times, for instance, to compute different relational values for different flow fluids and/or classes of flow fluids.

### Graphs

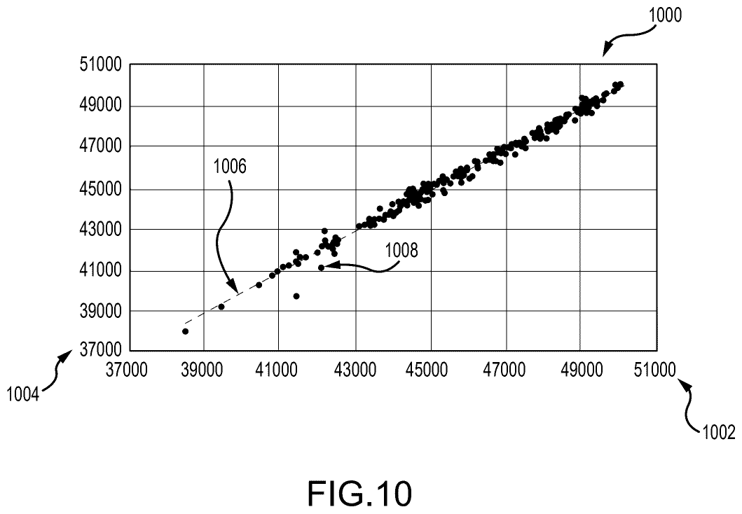

FIGS. 10-13 show graphs of embodiments of comparisons between inferred energy contents described in the specification and directly determined energy contents.

An exemplary embodiment of the inferential methods carried out by the measurement module 202, the inference module 204, and/or the response module 206 may be shown by showing first a determination of values in a CV inferential relationship and then a use of the relationship to determine an inferred CV.

In this exemplary embodiment, a test case is presented. In the test case, 200 fluids of different composition, according to the ISO10723, are used to determine the coefficients for relationships expressed in Eqs. (2)-(13). The relative composition of the substances within the flow fluid had ranges of composition described in Table 1.

TABLE 1
Composition Ranges

| | | | carbon | carbon | | | | |
|---|---|---|---|---|---|---|---|---|
| | methane | nitrogen | monoxide | dioxide | ethane | ethylene | propane | propylene |
| | (%) | (%) | (%) | (%) | (%) | (%) | (%) | (%) |
| Max (%) | 99.1 | 9.9 | 0.0 | 9.1 | 15.4 | 0.0 | 7.3 | 0.0 |
| Min (%) | 78.0 | 0.0 | 0.0 | 0.0 | 0.0 | 0.0 | 0.0 | 0.0 |
| | isobutane | butane | ipentane | pentane | hexane | heptane | hydrogen | helium |
| | (%) | (%) | (%) | (%) | (%) | (%) | (%) | (%) |
| Max (%) | 1.0 | 2.8 | 0.6 | 0.8 | 0.5 | 0.0 | 0.0 | 0.0 |
| Min (%) | 0.0 | 0.0 | 0.0 | 0.0 | 0.0 | 0.0 | 0.0 | 0.0 |
| | oxygen | water | argon | | | | | |
| | (%) | (%) | (%) | | | | | |
| Max (%) | 0.1 | 0.0 | 0.0 | | | | | |
| Min (%) | 0.0 | 0.0 | 0.0 | | | | | |

Using the NIST Refprop database 23 version 9.1, the properties of these gases were determined over a pressure (P) range of 1 to 3 bar and a temperature (T) range of 20° C. to 30° C. Conducting regression on this data yielded a set of coefficient constants that may be applied dynamically to sets of measured parameters as disclosed in this specification. The mass-based units may be kilojoules per kilogram (KJ/kg). The coefficient constants determined in terms of gas mixtures in mass units yielded in this test case are shown in Table 2.

TABLE 2
Coefficient Constants (Determined From Mass Units)

| | | | | | |
|---|---|---|---|---|---|
| a₁ | 1.2564E+05 | b₁ | 3.6445E+02 | c₁ | −9.8455E+03 |
| a₂ | 2.6929E+02 | b₂ | −6.8230E−01 | c₂ | 9.2347E+00 |
| a₃ | 7.9910E+02 | b₃ | 2.0611E+04 | c₃ | −4.1046E+01 |
| a₄ | −3.8362E−01 | b₄ | −1.0474E+02 | c₄ | 7.4090E−02 |

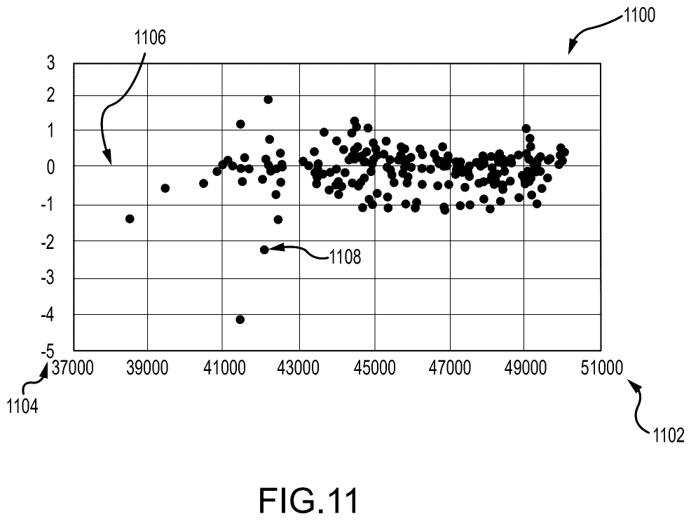

The results of using the coefficient constants, determined from mass unit quantities, in the relationships expressed by Eqs. (2)-(13) applied to 200 other random gas mixtures within the same parameters at which the constants were determined can be seen in FIGS. 10-11. The energy content metric used in the FIGS. 10-11 is Calorific Value.

FIG. 10 shows a graph 1000 of an embodiment of a comparison between inferred energy content values derived using mass units and energy content determined from direct methods. Graph 1000 has an abscissa 1002 that represents directly determined calorific values in units of kJ/kg, an ordinate 1004 that represents inferred calorific values in units of kJ/kg, a trendline 1006 that represents the comparison of the inferred calorific values and the determined calorific values, and a plurality of points 1008 that represent the comparison of the inferred calorific values and the determined calorific values. As can be seen, the results track relatively well. The trendline 1006 is determined as (inferred CV)=(directly determined CV)×1.0065−347.3. A trendline 1006 that is close to having a slope of one with an intercept that is less than one percent of the quantities measured shows a very strong correlation. The R-Squared value of the trendline is 0.989, also showing a strong correlation between inferred calorific value and directly determined calorific value.

FIG. 11 shows a graph 1100 of an embodiment of error in the inferred calorific values relative to the directly determined calorific values. Graph 1100 has an abscissa 1102 that represents the value of directly determined calorific value in kJ/kg, an ordinate 1104 that represents the percent error between the inferred calorific value and the directly determined calorific value, a zero error reference 1106, and a plurality of points 1108 that represent the comparison of directly determined calorific values to errors between the inferred calorific values and directly determined calorific values. The results yielded errors with a standard deviation of 0.60%. Of the 200 gases evaluated, 5 gases gave noticeably higher errors than the rest, suggesting that the error for inferring calorific value for most gases will be significantly less than the aforementioned standard deviation.

In another embodiment, coefficient constants can be determined in units of kilojoules per standard cubic meter (kJ/stdm³) at base conditions of 20° C. and 1.013 bar (hereinafter, “standard conditions”). Analyses of quantities as standard conditions were conducted similarly to the analyses applied for the mass unit coefficient constant determinations. The constants yielded by this determination are shown in table 3.

TABLE 3
Coefficient Constants (Determined At Base Conditions)

| | | | | | |
|---|---|---|---|---|---|
| a₁ | 1.3390E+05 | b₁ | 1.4427E+02 | c₁ | −7.7621E+03 |
| a₂ | 2.1595E+02 | b₂ | 6.5259E−01 | c₂ | 6.9745E+00 |
| a₃ | 2.1112E+02 | b₃ | 1.0602E+04 | c₃ | −6.5626E+00 |
| a₄ | 3.1515E+00 | b₄ | 5.3557E+00 | c₄ | −2.1600E−01 |

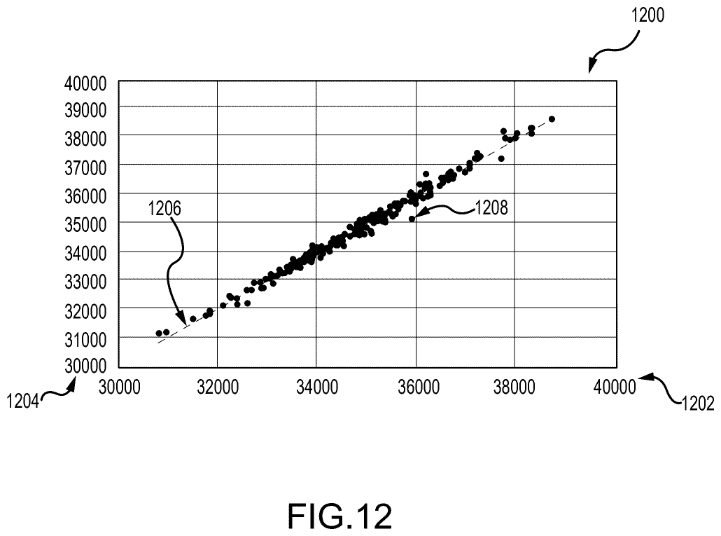

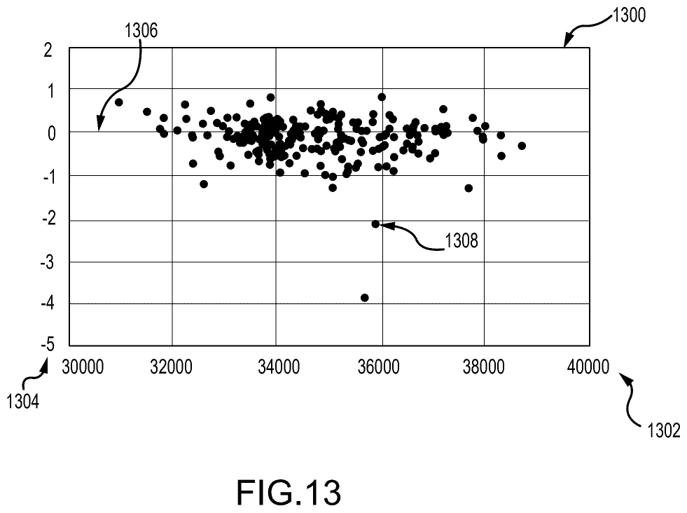

The results of using the coefficient constants, determined from standard condition quantities, in the relationships expressed by Eqs. (2)-(13) applied to 200 other random gas mixtures within the same parameters at which the constants were determined can be seen in FIGS. 12-13. The energy content metric used in FIGS. 12-13 is Calorific Value.

FIG. 12 shows a graph 1200 of an embodiment of a comparison between inferred energy content values inferred at standard conditions and energy content determined from direct methods. Graph 1200 has an abscissa 1202 that represents directly determined calorific values in units of kJ/stdm³, an ordinate 1204 that represents inferred calorific values in units of kJ/stdm³, a trendline 1206 that represents the comparison of the inferred calorific values and the determined calorific values, and a plurality of points 1208 that represent the comparison of the inferred calorific values and the determined calorific values. As can be seen, the results track relatively well. The trendline 1206 is determined as (inferred CV)=(directly determined CV)×0.981+626.17. A trendline 1206 that is close to having a slope of one shows a very strong correlation. The R-Squared value of the trendline is 0.9847, also showing a strong correlation between inferred calorific value and directly determined calorific value.

FIG. 13 shows a graph 1300 of an embodiment of error in the inferred calorific values relative to the directly determined calorific values. Graph 1300 has an abscissa 1302 that represents the value of directly determined calorific value in kJ/stdm³, an ordinate 1304 that represents the percent error between the inferred calorific value and the directly determined calorific value, a zero error reference 1306, and a plurality of points 1308 that represent the comparison of directly determined calorific values to errors between the inferred calorific values and directly determined calorific values. The results yielded errors with a standard deviation of 0.54%.

The graphs 1000-1300 show that inferential determinations of calorific value based on measured temperature (T), pressure (P), density (ρ), and viscosity (η) measurements and using the relationships (e.g. Eqs. (2)-(13)) expressed in this specification closely approximate calorific values that are directly determined from traditional direct methods that require equipment less typically found measuring fluid flow lines.

The detailed descriptions of the above embodiments are not exhaustive descriptions of all embodiments contemplated by the inventors to be within the scope of the present description. Indeed, persons skilled in the art will recognize that certain elements of the above-described embodiments may variously be combined or eliminated to create further embodiments, and such further embodiments fall within the scope and teachings of the present description. It will also be apparent to those of ordinary skill in the art that the above-described embodiments may be combined in whole or in part to create additional embodiments within the scope and teachings of the present description. When specific numbers representing parameter values are specified, the ranges between all of those numbers as well as ranges above and ranges below those numbers are contemplated and disclosed.

Thus, although specific embodiments are described herein for illustrative purposes, various equivalent modifications are possible within the scope of the present description, as those skilled in the relevant art will recognize. The teachings provided herein can be applied to other methods and apparatuses for inferring calorific values and not just to the embodiments described above and shown in the accompanying figures. Accordingly, the scope of the embodiments described above should be determined from the following claims.

We claim:

1. A method for operating a vibratory sensor, comprising:
flowing a fluid comprising a combustible component through the vibratory sensor, wherein the fluid is in contact with a vibratable element;
coupling a driver to the vibratable element;
coupling a response sensor to the vibratable element;
providing a vibratory signal to the driver, with meter electronics;
receiving a vibratory response signal from the response sensor, by the meter electronics;
processing, by the meter electronics, the vibratory response signal and measuring a density (ρ) of the fluid based on the processing;
using an inference module, in communication with the meter electronics, to use an inferential relationship between an inferred energy content and at least one measured quantity of the fluid, to yield an inferred energy content of the fluid, wherein the inference module does not account for thermal conductivity, heat capacity, wherein the using comprises:
receiving, by the inference module (204), at least one measured value of a type of the at least one measured quantity; and
inferring, by the inference module (204), the real-time inferred energy content from the inferential relationship and the at least one measured quantity,
wherein the inferential relationship has a density term (B) and one of the at least one measured value is the measured density (ρ) and the density term (B) comprises an inverse density term 1/ρ, the density term (B) representing an inverse relationship between the measured density (ρ) and the inferred energy content and wherein the measured density (ρ) is not a density of air (ρₐᵢᵣ), and wherein the inference module (204) does not account for any of viscosity (η), specific gravity, and the density of air (ρair) in the density term (B).

2. The method as claimed in claim 1, wherein the inferential relationship is a sum of a shift term (A), the density term (B), and a viscosity term (C).

3. The method as claimed in claim 2, wherein the at least one measured value further comprises a measured temperature (T) and a measured pressure (P) wherein the shift term (A) comprises a corresponding temperature and pressure dependent shift term coefficient (k₁(P,T)), the density term (B) comprises a corresponding temperature and pressure dependent density term coefficient (k₂(P,T)), and the viscosity term (C) comprises a corresponding temperature and pressure dependent viscosity term coefficient (k₃(P,T)).

4. The method as claimed in claim 3, wherein the inferential relationship is represented by the equation,
$$CV = {k}_{1} ( P , T ) + {k}_{2} ( P , T ) \times \frac{1}{ρ} + {k}_{3} ( P , T ) \times η ,$$
wherein CV comprises the inferred energy content, and wherein n is a viscosity of the fluid.

5. The method as claimed in claim 3, wherein the shift term coefficient (k₁(P, T)) is evaluated, by the inference module (204), using a relationship between the measured pressure (P), the measured temperature (T), and at least one predetermined shift coefficient constant of a plurality of predetermined coefficient constants, the density term coefficient (k₂(P,T)) is evaluated, by the inference module (204), using a relationship between the measured pressure (P), the measured temperature (T) and at least one predetermined density coefficient constant of the plurality of predetermined coefficient constants, and the viscosity term coefficient (k₃(P,T)) is evaluated, by the inference module (204), using a relationship between the measured pressure (P), the measured temperature (T) and at least one predetermined viscosity coefficient constant of the plurality of predetermined coefficient constants.

6. The method as claimed in claim 5, wherein the relationship between the measured pressure (P), the measured temperature (T) and at least one of the plurality of predetermined shift coefficient constant is represented by the equation, k₁(P,T)=[a₁+a₂(T−20)]+[a₃+a₄(T−20)]×P, the relationship between the measured pressure (P), the measured temperature (T) and the at least one predetermined density coefficient constant is represented by the equation, k₂(P,T)=[b₁+b₂(T−20)]+[b₃+b₄(T−20)]×P, the relationship between the measured pressure (P), the measured temperature (T) and the at least one predetermined viscosity coefficient constant is represented by the equation, k₃(P,T)=[c₁+c₂(T−20)]+[c₃+c₄(T−20)]×P, wherein a1-a4 are predetermined shift coefficient constants of the at least one predetermined shift coefficient constant, b1-b4 are predetermined density coefficient constants of the at least one predetermined density coefficient constant and c1-c4 are predetermined viscosity coefficient constants of the at least one predetermined viscosity coefficient constant.

7. The method as claimed in claim 3, the at least one measured value further comprising a measured inert content, wherein the measured inert content is a percent composition of carbon dioxide by volume (% CO₂), the inferential relationship further having an inert term (D), the inert term (D) accounting for the percent composition of carbon dioxide (% CO₂), the inert term (D) having a temperature (T) and pressure (P) dependent inert term coefficient (k₄(P,T)), wherein the inert term coefficient (k₄(P,T)) is determined using inert term coefficient constants.

8. The method as claimed in claim 7, the inert term coefficient (k₄(P,T)) being determined from the relationship, k₄(P,T)=[d₁+d₂(T−20)]+[d₃+d₄(T−20)]×P, the inferential relationship being
$$CV = {k}_{1} ( P , T ) + {k}_{2} ( P , T ) \times \frac{1}{ρ} + {k}_{3} ( P , T ) \times η + {k}_{4} ( P , T ) \times \% {CO}_{2} ,$$
wherein η is a viscosity of the fluid, and wherein d1-d4 are the inert term coefficient constants.

9. The method as claimed in claim 1, wherein the inferring, by the inference module (204), comprises inferring the inferred energy content of the fluid using predetermined coefficient constants associated with at least one class of fluids of which the fluid is a member.

10. The method as claimed in claim 1, further comprising measuring, by a pressure sensor (150), a measured pressure (P), wherein the receiving, by the inference module (204), comprises receiving the measured pressure (P).

11. A vibratory sensor, comprising:
a vibratable element in contact with a fluid comprising a combustible component and flowing through the vibratory sensor;
a driver in communication with a vibratable element;
a response sensor in communication with the vibratable element;
meter electronics operable to generate and provide a vibratory signal to the driver, and receive a vibratory response signal from the response sensor, and further operable to process the vibratory response signal and measure a density (ρ) of the fluid based on the processed vibratory response;
an inference module (204), in communication with the meter electronics, configured to use an inferential relationship between an inferred energy content and at least one measured quantity of the fluid, the inferential relationship yielding an inferred energy content, wherein the inference module does not account for thermal conductivity or heat capacity, and wherein the inference module (204) is further configured to:
receive at least one measured value of a type of the at least one measured quantity; and
infer the inferred energy content, in real time, from the inferential relationship and the at least one measured quantity, wherein the inferential relationship has a density term (B) and one of the at least one measured value is the measured density (ρ) and the density term (B) comprises an inverse density term 1/ρ, the density term (B) representing an inverse relationship between the measured density (ρ) and the inferred energy content and wherein the measured density (ρ) is not a density of air (ρₐᵢᵣ), wherein the inference module (204) does not account for any of viscosity (η), specific gravity, and the density of air (ρair) in the density term (B).

12. A vibratory sensor as claimed in claim 11, wherein the inferential relationship is a sum of a shift term (A), the density term (B), and a viscosity term (C).

13. A vibratory sensor as claimed in claim 12, wherein the at least one measured value further comprises a measured temperature (T) and a measured pressure (P) wherein the shift term (A) comprises a corresponding temperature and pressure dependent shift term coefficient (k₁(P,T)), the density term (B) comprises a corresponding temperature and pressure dependent density term coefficient (k₂(P,T)), and the viscosity term (C) comprises a corresponding temperature and pressure dependent viscosity term coefficient (k₃(P,T)).

14. A vibratory sensor as claimed in claim 13, wherein the inferential relationship is represented by the equation,
$$CV = {k}_{1} ( P , T ) + {k}_{2} ( P , T ) \times \frac{1}{ρ} + {k}_{3} ( P , T ) \times η$$
wherein CV comprises the inferred energy content, and wherein η is a viscosity of the fluid.

15. A vibratory sensor as claimed in claim 13, wherein the shift term coefficient (k₁(P,T)) is evaluated, by the inference module (204), using a relationship between the measured pressure (P), the measured temperature (T), and at least one predetermined shift coefficient constant of a plurality of predetermined coefficient constants, the density term coefficient (k₂(P,T)) is evaluated, by the inference module (204), using a relationship between the measured pressure (P), the measured temperature (T) and at least one predetermined density coefficient constant of the plurality of predetermined coefficient constants, and the viscosity term coefficient (k₃(P,T)) is evaluated, by the inference module (204), using a relationship between the measured pressure (P), the measured temperature (T) and at least one predetermined viscosity coefficient constant of the plurality of predetermined coefficient constants.

16. The method as claimed in claim 1, further comprising:
determining, by the inference module (204), the inferential relationship by analyzing a relationship between known measurements of at least one measured energy content of at least one fluid and at least one corresponding measured value of a same type as the at least one measured quantity.

17. A vibratory sensor as claimed in claim 11 wherein the inference module is further configured to determine the inferential relationship by analyzing a relationship between known measurements of at least one measured energy content of at least one fluid and at least one corresponding measured value of a same type as the at least one measured quantity.
